SEQUENCE LISTING (45 sequences)

SEQ ID NO: 1 - protein, 494 aa, Geobacillus sp.
MKMPAKTGKEYMERLKQAKSSVYIHGEKVEDVTVHPAFRNVVRSMAALYDRQYEKPEKMLYRSPTTGQPVGMTFIQPTTIDELIARREATQEWARMSAGMMGRSPDYLNAEVMAMGIANDLFAEDDPMFAENAKNYYEYAREHDISLTHTLIHPQMNRAKALHEQNDADVPLHLVERRKDGIIVSGIRLLATQGGITDEILVFPSTVKKATSGEDPYALAFAIPNNTPGVKFICREAFDYGRSAWDHPLASRFEEGDAIVSFENVFVPWERVFVCGNSSICNRTFRETNAVVHMSHQVVAKNIVKTEFLLGVTLCLIEAIGIGEFQHVKDKGAEIMLVLETMKSHLYRAEHNAKRDRWGTMTPDFAALDAARNWYPRIYPRLAEIIRILGASGLMAIPTEADFQHEEIGDIVRRAMQGATVDGYERVQLFRLAWDLTMSAFGARQTHYEYYFFGDPVRMGMAYFDGYEKEPYKQFVREFLRGAKSVFIPADNKH

SEQ ID NO: 2 - protein, 520 aa, E. coli
MKPEDFRASTQRPFTGEEYLKSLQDGREIYIYGERVKDVTTHPAFRNAAASVAQLYDALHKPEMQDSLCWNTDTGSGGYTHKFFRVAKSADDLRHERDAIAEWSRLSYGWMGRTPDYKAAFGCALGGTPGFYGQFEQNARNWYTRIQETGLYFNHAIVNPPIDRHLPTDKVKDVYIKLEKETDAGIIVSGAKVVATNSALTHYNMIGFGSAQVMGENPDFALMFVAPMDADGVKLISRASYEMVAGATGSPYDYPLSSRFDENDAILVMDNVLIPWENVLLYRDFDRCRRWTMEGGFARMYPLQACVRLAVKLDFITALLKKSLECTGTLEFRGVQADLGEVVAWRNTFWALSDSMCSEATPWVNGAYLPDHAALQTYRVLAPMAYAKIKNIIERNVTSGLIYLPSSARDLNNPQIDQYLAKYVRGSNGMDHVQRIKILKLMWDAIGSEFGGRHELYEINYSGSQDEIRLQCLRQAQSSGNMDKMMAMVDRCLSEYDQNGWTVPHLHNNDDINMLDKLLK

SEQ ID NO: 3 - protein, 481 aa, Thermus thermophilus
MARTGAEYIEALKTRPPNLWYKGEKVEDPTTHPVFRGIVRTMAALYDLQHDPRYREVLTYEEEGKRHGMSFLIPKTKEDLKRRGQAYKLWADQNLGMMGRSPDYLNAVVMAYAASADYFGEFAENVRNYYRYLRDQDLATTHALTNPQVNRARPPSGQPDPYIPVGVVKQTEKGIVVRGARMTATFPLADEVLIFPSTLLQAGSEKYALAFALPTSTPGLHFVCREALVGGDSPFDHPLSSRVEEMDCLVIFDDVLVPWERVFILGNVELCNNAYAATGALNHMAHQVVALKTAKTEAFLGVAALMAEGIGADVYGHVQEKIAEIIVYLEAMRAFWTRAEEEAKENAYGLLVPDRGALDGARNLYPRLYPRIREILEQIGASGLITLPSEKDFKGPLGPFLEKFLQGAALEAKERVALFRLAWDMTLSGFGARQELYERFFFGDPVRMYQTLYNVYNKEPYKERIRAFLKESLKVFEEVQA

SEQ ID NO: 4 - protein, 527 aa, Streptomyces globisporus
MPHGAEREASPAEESAGTRPLTGEEYLESLRDAREVYLDGSRVKDVTAHPAFHNPARMTARLYDSLHDPAQKAVLTAPTDAGDGFTHRFFTAPRSVDDLVKDQAAIASWARKSYGWMGRSPDYKASFLGTLGANADFYEPFADNARRWYRESQEKVLYWNHAFLHPPVDRSLPADEVGDVFIHVERETDAGLVVSGAKVVATGSALTHAAFISHWGLPIKDRKFALVATVPMDADGLKVICRPSYSANAATTGSPFDNPLSSRLDENDAILVLDQVLIPWENVFVYGNLGKVHLLAGQSGMIERATFHGCTRLAVKLEFIAGLLAKALDITGAKDFRGVQTRLGEVLAWRNLFWSLSDAAARNPVPWKNGTLLPNPQAGMAYRWFMQIGYPRVLEIVQQDVASGLMYVNSSTEDFRNPETGPYLEKYLRGSDGAGAVERVKVMKLLWDAVGSDFGGRHELYERNYSGNHENTRIELLLSQTASGKLDSYMDFAQACMDEYDLDGWTAPDLESFHAMRSASRDLLGGL

SEQ ID NO: 5 - protein, 490 aa, Clostridium aminobutyricum
MLMTAEQYIESLRKLNTRVYMFGEKIENWVDHPMIRPSINCVRMTYELAQDPQYADLMTTKSNLIGKTINRFANLHQSTDDLRKKVKMQRLLGQKTASCFQRCVGMDAFNAVFSTTYEIDQKYGTNYHKNFTEYLKYIQENDLIVDGAMTDPKGDRGLAPSAQKDPDLFLRIVEKREDGIVVRGAKAHQTGSINSHEHIIMPTIAMTEADKDYAVSFACPSDADGLFMIYGRQSCDTRKMEEGADIDLGNKQFGGQEALVVFDNVFIPNDRIFLCQEYDFAGMMVERFAGYHRQSYGGCKVGVGDVVIGAAALAADYNGAQKASHVKDKLIEMTHLNETLYCCGIACSAEGYPTAAGNYQIDLLLANVCKQNITRFPYEIVRLAEDIAGGLMVTMPSEADFKSETVVGRDGETIGDFCNKFFAAAPTCTTEERMRVLRFLENICLGASAVGYRTESMHGAGSPQAQRIMIARQGNINAKKELAKAIAGIK

SEQ ID NO: 6 - protein, 515 aa, Burkholderai cepacia
MRTGKQYLESLNDGRVVWVGNEKIDNVATHPLTRDYAERVAQFYDLHHRPDLQDVLTFVDADGVRRSRQWQDPKDAAGLRVKRKYHETILREIAAGSYGRLPDAHNYTFTTYADDPEVWEKQSIGAEGRNLTQNIHNFLKLLREKDLNCPLNFVDPQTDRSSDAAQARSPNLRIVEKTDDGIIVNGVKAVGTGIAFGDYMHIGCLYRPGIPGEQVIFAAIPTNTPGVTVFCRESTVKNDPAEHPLASQGDELDSTTVFDNVFIPWEQVFHIGNPEHAKLYPQRIFDWVHYHILIRQVLRAELIVGLAILITEHIGTSKLPTVSARVAKLVAFHLAMQAHLIASEETGFHTKGGRYKPNPLIYDFGRAHFLQNQMSVMYELLDLAGRSSLMIPSEGQWDDSQSGQWFVKLNNGPKGNPRERVQIGRVIRDLYLTDWGGRQFMFENFNGTPLFAVFAATMTRDDMSAAGTYGKFASQVCGIEFGGAEPTAYAATADYAKALDKGLAPEPAAAESATS

SEQ ID NO: 7 - protein, 517 aa, Cupriavidus necator
MIRTGKQYLESLNDGRNVWVGNEKIDNVATHPKTRDYAQRHADFYDLHHRPDLQDVMTFVDKDGERRTMQWFGHYDKEQLRRKRKYHETIMREMAGASFPRTPDVNNYVLQTYIDDPSPWETQTIGAEGKVKAKNIVDFVNFAKKHDLNCAPQFVDPQMDRSNPDAQQRSPGLRVIEKNDKGIVVSGVKAIGTGVAFADWIHIGVFFRPGIPGDQIIFAATPVNTPGVTIVCRESVVKEDPIEHPLASQGDELDGMTVFDNVFIPWSHVFHLGNPEHAKLYPQRVFDWLHYHALIRQSVRAELMAGLAILITEHIGTNKIPAVQTRVAKLIGFHQAMLAHIVASEELGFHTPGGAYKPNILIYDFGRALYLENFSQMIYELVDLSGRSALIFASEDQWNDEALNGWFERMNNGPVGQPHDRVKIGRVIRDLFLTDWGNRLFVFENFNGTPLQAIRMLTMQRAEFSAAGPYGTLARKVCGIELTEGHDSEYKATAGYAQALDSARHQEKLALSGTMTV

SEQ ID NO: 8 - protein, 416 aa, Oscillatoria sp. PCC 6506
MGSSHHHHHHSSGLVPRGSHMASMTGGQQMGRGSEFDFAWNSQQIQFRKKVIQFAQQSLISDLIKNDKEEIFNRDAWQKCSEFGVHGWPIPARYGGQELDILTTAYALQGLGYGCKDNGLIFAMNAHIWACEMPLLTFGTEEQKEKYLPLLCRGGWIASHAATEPQAGSDIYSLKTTAQKDGDKYILNGYKHYVTNGTIADLFIIFATIDPSLGKEGLTTFMIEKDTPGLILSKPISKMGMRTAEVPELRLENCEVSAANRLGEEGTGLAIFNHSMEWERGFILAAAVGTMERLLEQSIRYARSHKQFGQAIGKFQLVANKLVEMKLRLENAKAYLYKVAWMKENKQMALLEASMANLYISEAWVQSCLEAIEIHGAYGYLTNTELERELRDAIASKFYSGTSEIQRVVIAKFLGL

SEQ ID NO: 9 - protein, 410 aa, Paraburkholderia phymatum
MAHHHHHHMLAANLIDPHGALAWPFFEARHRELAAGIEAWATQHLACVQHDDTDTTCRKLVRALGEAGWLKYGVGGAQYGGHGDTIDTRAVCLLRETLANHDGLADFALAMQGLGSGAITLAGTHEQKIRYLPRVSKGEAIAAFALSEPDAGSDVAAMSLQARAEGDCYVIDGDKTWISNGGIADFYVVFARTGEAPGARGISAFIVDADTPGLQIAERIDVIAPHPLARLHFDSARVPRSQMLGAPGEGFKIAMRTLDVFRTSVAAASLGFARRALQEGLARAASRKMFGQTLGDFQLTQTKLAQMALTIDSSALLVYRAAWLRDQGENVTREAAMAKWHASEGAQQVIDAAVQLWGGMGVQSGTTVERLYREIRALRIYEGATEVQQLIVGRDLLKAHAAQRQQERAS

SEQ ID NO: 10 - protein, 517 aa, Ralstonia pickettii
MIRTGTQYLESLNDGRNVWVGNEKIDNVATHPKTRDYAQRHADFYDLHHRPDLQDVMTYIDEGGQRRAMQWFGHRDKEQLRRKRKYHETVMREMAGASFPRTPDVNNYVLTTYIDDPAPWETQSIGDDGHIKAGKIVDFIRYAREHDLNCAPQFVDPQMDRSNPDAQERSPGLRVVEKNEKGIVVNGVKAIGTGVAFADWIHIGVFFRPGIPGDQVIFAATPVNTPGVTIVCRESLVKDDKVEHPLAAQGDELDGMTVFENVFIPWSHVFHIGNPNHAKLYPQRVFDWLHYHALIRQMVRAELVAGLAVLITEHIGTNKIPAVQTRVAKLIGFHQAMLAHLIASEELGFHTPGGHYKPNILIYDFGRALYLENFSQMIYELVDLSGRSALIFASEDQWNDDKLNGWFERMNNGPVGRPHDRVKIGRVIRDLFLTDWGSRLFVFENFNGTPLQGIRMLTMQRAEFSGSGPYGKLARQVCGIDSAVTDDTEYRKTADYAKALDAARHQEEVALAGAMAI

SEQ ID NO: 11 - protein, 494 aa, Geobacillus sp.
MKMPAKTGKEYMERLKQAKSSVYIHGEKVEDVTVHPAFRNVVRSMAALYDRQYEKPEKMLYRSPTTGQPVGMTFIQPTTIDELIARREATQEWARMSAGMMGRSPDYLNAEVMAMGIANDLFAEDDPMFAENAKNYYEYAREHDISLTHTLIHPQMNRAKALHEQNDADVPLHLVERRKDGIIVSGIRLLATQGGITDEILVFPSTLKKATSGEDPYALAFAIPNNTPGVKFICREAFDYGRSAWDHPLASRFEEGDAIVSFENVFVPWERVFVCGNSSICNRTFRETNAVVHMSHQVVAKNIVKTEFLLGVTLCLIEAIGIGEFQHVKDKGAEIMLVLETMKSHLYRAEHNAKRDRWGTMTPDFAALDAARNWYPRIYPRLAEIIRILGASGLMAIPTEADFQHEEIGDIVRRAMQGATVDGYERVQLFRLAWDLTMSAFGARQTHYEYYFFGDPVRMGMAYFDGYEKEPYKQFVREFLRGAKSVFIPADNKH

SEQ ID NO: 12 - protein, 494 aa, Geobacillus sp
MKMPAKTGKEYMERLKQAKSSVYIHGEKVEDVTVHPAFRNVVRSMAALYDRQYEKPEKMLYRSPTTGQPVGMTFIQPTTIDELIARREATQEWARMSAGMMGRSPDYLNAEVMAMGIANDLFAEDDPMFAENAKNYYEYAREHDISLTHTLIHPQMNRAKALHEQNDADVPLHLVERRKDGIIVSGIRLLATQGGITDEILVFPSTTKKATSGEDPYALAFAIPNNTPGVKFICREAFDYGRSAWDHPLASRFEEGDAIVSFENVFVPWERVFVCGNSSICNRTFRETNAVVHMSHQVVAKNIVKTEFLLGVTLCLIEAIGIGEFQHVKDKGAEIMLVLETMKSHLYRAEHNAKRDRWGTMTPDFAALDAARNWYPRIYPRLAEIIRILGASGLMAIPTEADFQHEEIGDIVRRAMQGATVDGYERVQLFRLAWDLTMSAFGARQTHYEYYFFGDPVRMGMAYFDGYEKEPYKQFVREFLRGAKSVFIPADNKH

SEQ ID NO: 13 - protein, 494 aa, Geobacillus sp.
MKMPAKTGKEYMERLKQAKSSVYIHGEKVEDVTVHPAFRNVVRSMAALYDRQYEKPEKMLYRSPTTGQPVGMTFIQPTTIDELIARREATQEWARMSAGMMGRSPDYLNAEVMAMGIANDLFAEDDPMFAENAKNYYEYAREHDISLTHTLIHPQMNRAKALHEQNDADVPLHLVERRKDGIIVSGIRLLATQGGITDEILVFPSTQKKATSGEDPYALAFAIPNNTPGVKFICREAFDYGRSAWDHPLASRFEEGDAIVSFENVFVPWERVFVCGNSSICNRTFRETNAVVHMSHQVVAKNIVKTEFLLGVTLCLIEAIGIGEFQHVKDKGAEIMLVLETMKSHLYRAEHNAKRDRWGTMTPDFAALDAARNWYPRIYPRLAEIIRILGASGLMAIPTEADFQHEEIGDIVRRAMQGATVDGYERVQLFRLAWDLTMSAFGARQTHYEYYFFGDPVRMGMAYFDGYEKEPYKQFVREFLRGAKSVFIPADNKH

SEQ ID NO: 14 - protein, 494 aa, Geobacillus sp.
MKMPAKTGKEYMERLKQAKSSVYIHGEKVEDVTVHPAFRNVVRSMAALYDRQYEKPEKMLYRSPTTGQPVGMTFIQPTTIDELIARREATQEWARMSAGMMGRSPDYLNAEVMAMGIANDLFAEDDPMFAENAKNYYEYAREHDISLTHTLIHPQMNRAKALHEQNDADVPLHLVERRKDGIIVSGIRLLATQGGITDEILVFPSTGKKATSGEDPYALAFAIPNNTPGVKFICREAFDYGRSAWDHPLASRFEEGDAIVSFENVFVPWERVFVCGNSSICNRTFRETNAVVHMSHQVVAKNIVKTEFLLGVTLCLIEAIGIGEFQHVKDKGAEIMLVLETMKSHLYRAEHNAKRDRWGTMTPDFAALDAARNWYPRIYPRLAEIIRILGASGLMAIPTEADFQHEEIGDIVRRAMQGATVDGYERVQLFRLAWDLTMSAFGARQTHYEYYFFGDPVRMGMAYFDGYEKEPYKQFVREFLRGAKSVFIPADNKH

SEQ ID NO: 15 - protein, 494 aa, Geobacillus sp.
MKMPAKTGKEYMERLKQAKSSVYIHGEKVEDVTVHPAFRNVVRSMAALYDRQYEKPEKMLYRSPTTGQPVGMTFIQPTTIDELIARREATQEWARMSAGMMGRSPDYLNAEVMAMGIANDLFAEDDPMFAENAKNYYEYAREHDISLTHTLIHPQMNRAKALHEQNDADVPLHLVERRKDGIIVSGIRLLATQGGITDEILVFPSMVKKATSGEDPYALAFAIPNNTPGVKFICREAFDYGRSAWDHPLASRFEEGDAIVSFENVFVPWERVFVCGNSSICNRTFRETNAVVHMSHQVVAKNIVKTEFLLGVTLCLIEAIGIGEFQHVKDKGAEIMLVLETMKSHLYRAEHNAKRDRWGTMTPDFAALDAARNWYPRIYPRLAEIIRILGASGLMAIPTEADFQHEEIGDIVRRAMQGATVDGYERVQLFRLAWDLTMSAFGARQTHYEYYFFGDPVRMGMAYFDGYEKEPYKQFVREFLRGAKSVFIPADNKH

SEQ ID NO: 16 - protein, 494 aa, Geobacillus sp.
MKMPAKTGKEYMERLKQAKSSVYIHGEKVEDVTVHPAFRNVVRSMAALYDRQYEKPEKMLYRSPTTGQPVGMTFIQPTTIDELIARREATQEWARMSAGMMGRSPDYLNAEVMAMGIANDLFAEDDPMFAENAKNYYEYAREHDISLTHTLIHPQMNRAKALHEQNDADVPLHLVERRKDGIIVSGIRLLATQGGITDEILVFPSAVKKATSGEDPYALAFAIPNNTPGVKFICREAFDYGRSAWDHPLASRFEEGDAIVSFENVFVPWERVFVCGNSSICNRTFRETNAVVHMSHQVVAKNIVKTEFLLGVTLCLIEAIGIGEFQHVKDKGAEIMLVLETMKSHLYRAEHNAKRDRWGTMTPDFAALDAARNWYPRIYPRLAEIIRILGASGLMAIPTEADFQHEEIGDIVRRAMQGATVDGYERVQLFRLAWDLTMSAFGARQTHYEYYFFGDPVRMGMAYFDGYEKEPYKQFVREFLRGAKSVFIPADNKH

SEQ ID NO: 17 - protein, 494 aa, Geobacillus sp.
MKMPAKTGKEYMERLKQAKSSVYIHGEKVEDVTVHPAFRNVVRSMAALYDRQYEKPEKMLYRSPTTGQPVGMTFIQPTTIDELIARREATQEWARMSAGMMGRSPDYLNAEVMAMGIANDLFAEDDPMFAENAKNYYEYAREHDISLTHTLIHPQMNRAKALHEQNDADVPLHLVERRKDGIIVSGIRLLATQGGITDEILVFPSTVRKATSGEDPYALAFAIPNNTPGVKFICREAFDYGRSAWDHPLASRFEEGDAIVSFENVFVPWERVFVCGNSSICNRTFRETNAVVHMSHQVVAKNIVKTEFLLGVTLCLIEAIGIGEFQHVKDKGAEIMLVLETMKSHLYRAEHNAKRDRWGTMTPDFAALDAARNWYPRIYPRLAEIIRILGASGLMAIPTEADFQHEEIGDIVRRAMQGATVDGYERVQLFRLAWDLTMSAFGARQTHYEYYFFGDPVRMGMAYFDGYEKEPYKQFVREFLRGAKSVFIPADNKH

SEQ ID NO: 18 - protein, 494 aa, Geobacillus sp.
MKMPAKTGKEYMERLKQAKSSVYIHGEKVEDVTVHPAFRNVVRSMAALYDRQYEKPEKMLYRSPTTGQPVGMTFIQPTTIDELIARREATQEWARMSAGMMGRSPDYLNAEVMAMGIANDLFAEDDPMFAENAKNYYEYAREHDISLTHTLIHPQMNRAKALHEQNDADVPLHLVERRKDGIIVSGIRLLATQGGITDEILVFPSTTRKATSGEDPYALAFAIPNNTPGVKFICREAFDYGRSAWDHPLASRFEEGDAIVSFENVFVPWERVFVCGNSSICNRTFRETNAVVHMSHQVVAKNIVKTEFLLGVTLCLIEAIGIGEFQHVKDKGAEIMLVLETMKSHLYRAEHNAKRDRWGTMTPDFAALDAARNWYPRIYPRLAEIIRILGASGLMAIPTEADFQHEEIGDIVRRAMQGATVDGYERVQLFRLAWDLTMSAFGARQTHYEYYFFGDPVRMGMAYFDGYEKEPYKQFVREFLRGAKSVFIPADNKH

SEQ ID NO: 19 - protein, 494 aa, Geobacillus sp.
MKMPAKTGKEYMERLKQAKSSVYIHGEKVEDVTVHPAFRNVVRSMAALYDRQYEKPEKMLYRSPTTGQPVGMTFIQPTTIDELIARREATQEWARMSAGMMGRSPDYLNAEVMAMGIANDLFAEDDPMFAENAKNYYEYAREHDISLTHTLIHPQMNRAKALHEQNDADVPLHLVERRKDGIIVSGIRLLATQGGITDEILVFPSMTKKATSGEDPYALAFAIPNNTPGVKFICREAFDYGRSAWDHPLASRFEEGDAIVSFENVFVPWERVFVCGNSSICNRTFRETNAVVHMSHQVVAKNIVKTEFLLGVTLCLIEAIGIGEFQHVKDKGAEIMLVLETMKSHLYRAEHNAKRDRWGTMTPDFAALDAARNWYPRIYPRLAEIIRILGASGLMAIPTEADFQHEEIGDIVRRAMQGATVDGYERVQLFRLAWDLTMSAFGARQTHYEYYFFGDPVRMGMAYFDGYEKEPYKQFVREFLRGAKSVFIPADNKH

SEQ ID NO: 20 - protein, 494 aa, Geobacillus sp.
MKMPAKTGKEYMERLKQAKSSVYIHGEKVEDVTVHPAFRNVVRSMAALYDRQYEKPEKMLYRSPTTGQPVGMTFIQPTTIDELIARREATQEWARMSAGMMGRSPDYLNAEVMAMGIANDLFAEDDPMFAENAKNYYEYAREHDISLTHTLIHPQMNRAKALHEQNDADVPLHLVERRKDGIIVSGIRLLATQGGITDEILVFPSATKKATSGEDPYALAFAIPNNTPGVKFICREAFDYGRSAWDHPLASRFEEGDAIVSFENVFVPWERVFVCGNSSICNRTFRETNAVVHMSHQVVAKNIVKTEFLLGVTLCLIEAIGIGEFQHVKDKGAEIMLVLETMKSHLYRAEHNAKRDRWGTMTPDFAALDAARNWYPRIYPRLAEIIRILGASGLMAIPTEADFQHEEIGDIVRRAMQGATVDGYERVQLFRLAWDLTMSAFGARQTHYEYYFFGDPVRMGMAYFDGYEKEPYKQFVREFLRGAKSVFIPADNKH

SEQ ID NO: 21 - protein, 481 aa, Thermus thermophilus
MARTGAEYIEALKTRPPNLWYKGEKVEDPTTHPVFRGIVRTMAALYDLQHDPRYREVLTYEEEGKRHGMSFLIPKTKEDLKRRGQAYKLWADQNLGMMGRSPDYLNAVVMAYAASADYFGEFAENVRNYYRYLRDQDLATTHALTNPQVNRARPPSGQPDPYIPVGVVKQTEKGIVVRGARMTATFPLADEVLIFPSTTLQAGSEKYALAFALPTSTPGLHFVCREALVGGDSPFDHPLSSRVEEMDCLVIFDDVLVPWERVFILGNVELCNNAYAATGALNHMAHQVVALKTAKTEAFLGVAALMAEGIGADVYGHVQEKIAEIIVYLEAMRAFWTRAEEEAKENAYGLLVPDRGALDGARNLYPRLYPRIREILEQIGASGLITLPSEKDFKGPLGPFLEKFLQGAALEAKERVALFRLAWDMTLSGFGARQELYERFFFGDPVRMYQTLYNVYNKEPYKERIRAFLKESLKVFEEVQA

SEQ ID NO: 22 - protein, 527 aa, Streptomyces globisporus
MPHGAEREASPAEESAGTRPLTGEEYLESLRDAREVYLDGSRVKDVTAHPAFHNPARMTARLYDSLHDPAQKAVLTAPTDAGDGFTHRFFTAPRSVDDLVKDQAAIASWARKSYGWMGRSPDYKASFLGTLGANADFYEPFADNARRWYRESQEKVLYWNHAFLHPPVDRSLPADEVGDVFIHVERETDAGLVVSGAKVVATGSALTHAAFISHTGLPIKDRKFALVATVPMDADGLKVICRPSYSANAATTGSPFDNPLSSRLDENDAILVLDQVLIPWENVFVYGNLGKVHLLAGQSGMIERATFHGCTRLAVKLEFIAGLLAKALDITGAKDFRGVQTRLGEVLAWRNLFWSLSDAAARNPVPWKNGTLLPNPQAGMAYRWFMQIGYPRVLEIVQQDVASGLMYVNSSTEDFRNPETGPYLEKYLRGSDGAGAVERVKVMKLLWDAVGSDFGGRHELYERNYSGNHENTRIELLLSQTASGKLDSYMDFAQACMDEYDLDGWTAPDLESFHAMRSASRDLLGGL

SEQ ID NO: 23 - protein, 490 aa, Clostridium aminobutyricum
MLMTAEQYIESLRKLNTRVYMFGEKIENWVDHPMIRPSINCVRMTYELAQDPQYADLMTTKSNLIGKTINRFANLHQSTDDLRKKVKMQRLLGQKTASCFQRCVGMDAFNAVFSTTYEIDQKYGTNYHKNFTEYLKYIQENDLIVDGAMTDPKGDRGLAPSAQKDPDLFLRIVEKREDGIVVRGAKAHQTGSINSHEHIIMPTTAMTEADKDYAVSFACPSDADGLFMIYGRQSCDTRKMEEGADIDLGNKQFGGQEALVVFDNVFIPNDRIFLCQEYDFAGMMVERFAGYHRQSYGGCKVGVGDVVIGAAALAADYNGAQKASHVKDKLIEMTHLNETLYCCGIACSAEGYPTAAGNYQIDLLLANVCKQNITRFPYEIVRLAEDIAGGLMVTMPSEADFKSETVVGRDGETIGDFCNKFFAAAPTCTTEERMRVLRFLENICLGASAVGYRTESMHGAGSPQAQRIMIARQGNINAKKELAKAIAGIK

SEQ ID NO: 24 - protein, 515 aa, Burkholderia cepacia
MRTGKQYLESLNDGRVVWVGNEKIDNVATHPLTRDYAERVAQFYDLHHRPDLQDVLTFVDADGVRRSRQWQDPKDAAGLRVKRKYHETILREIAAGSYGRLPDAHNYTFTTYADDPEVWEKQSIGAEGRNLTQNIHNFLKLLREKDLNCPLNFVDPQTDRSSDAAQARSPNLRIVEKTDDGIIVNGVKAVGTGIAFGDYMHIGCTYRPGIPGEQVIFAAIPTNTPGVTVFCRESTVKNDPAEHPLASQGDELDSTTVFDNVFIPWEQVFHIGNPEHAKLYPQRIFDWVHYHILIRQVLRAELIVGLAILITEHIGTSKLPTVSARVAKLVAFHLAMQAHLIASEETGFHTKGGRYKPNPLIYDFGRAHFLQNQMSVMYELLDLAGRSSLMIPSEGQWDDSQSGQWFVKLNNGPKGNPRERVQIGRVIRDLYLTDWGGRQFMFENFNGTPLFAVFAATMTRDDMSAAGTYGKFASQVCGIEFGGAEPTAYAATADYAKALDKGLAPEPAAAESATS

SEQ ID NO: 25 - protein, 517 aa, Cupriavidus necator
MIRTGKQYLESLNDGRNVWVGNEKIDNVATHPKTRDYAQRHADFYDLHHRPDLQDVMTFVDKDGERRTMQWFGHYDKEQLRRKRKYHETIMREMAGASFPRTPDVNNYVLQTYIDDPSPWETQTIGAEGKVKAKNIVDFVNFAKKHDLNCAPQFVDPQMDRSNPDAQQRSPGLRVIEKNDKGIVVSGVKAIGTGVAFADWIHIGTFFRPGIPGDQIIFAATPVNTPGVTIVCRESVVKEDPIEHPLASQGDELDGMTVFDNVFIPWSHVFHLGNPEHAKLYPQRVFDWLHYHALIRQSVRAELMAGLAILITEHIGTNKIPAVQTRVAKLIGFHQAMLAHIVASEELGFHTPGGAYKPNILIYDFGRALYLENFSQMIYELVDLSGRSALIFASEDQWNDEALNGWFERMNNGPVGQPHDRVKIGRVIRDLFLTDWGNRLFVFENFNGTPLQAIRMLTMQRAEFSAAGPYGTLARKVCGIELTEGHDSEYKATAGYAQALDSARHQEKLALSGTMTV

SEQ ID NO: 26 - protein, 416 aa, Oscillatoria sp. PCC 6506
MGSSHHHHHHSSGLVPRGSHMASMTGGQQMGRGSEFDFAWNSQQIQFRKKVIQFAQQSLISDLIKNDKEEIFNRDAWQKCSEFGVHGWPIPARYGGQELDILTTAYALQGLGYGCKDNGLIFAMNAHIWACEMPLLTFGTEEQKEKYLPLLCRGGWIASHAATEPQAGSDIYSLKTTAQKDGDKYILNGYKHYVTNGTIADLFIIFATTDPSLGKEGLTTFMIEKDTPGLILSKPISKMGMRTAEVPELRLENCEVSAANRLGEEGTGLAIFNHSMEWERGFILAAAVGTMERLLEQSIRYARSHKQFGQAIGKFQLVANKLVEMKLRLENAKAYLYKVAWMKENKQMALLEASMANLYISEAWVQSCLEAIEIHGAYGYLTNTELERELRDAIASKFYSGTSEIQRVVIAKFLGL

SEQ ID NO: 27 - protein, 410 aa, Paraburkholderia phymatum
MAHHHHHHMLAANLIDPHGALAWPFFEARHRELAAGIEAWATQHLACVQHDDTDTTCRKLVRALGEAGWLKYGVGGAQYGGHGDTIDTRAVCLLRETLANHDGLADFALAMQGLGSGAITLAGTHEQKIRYLPRVSKGEAIAAFALSEPDAGSDVAAMSLQARAEGDCYVIDGDKTWISNGGIADFYVVFARMGEAPGARGISAFIVDADTPGLQIAERIDVIAPHPLARLHFDSARVPRSQMLGAPGEGFKIAMRTLDVFRTSVAAASLGFARRALQEGLARAASRKMFGQTLGDFQLTQTKLAQMALTIDSSALLVYRAAWLRDQGENVTREAAMAKWHASEGAQQVIDAAVQLWGGMGVQSGTTVERLYREIRALRIYEGATEVQQLIVGRDLLKAHAAQRQQERAS

SEQ ID NO: 28 - protein, 517 aa, Ralstonia pickettii
MIRTGTQYLESLNDGRNVWVGNEKIDNVATHPKTRDYAQRHADFYDLHHRPDLQDVMTYIDEGGQRRAMQWFGHRDKEQLRRKRKYHETVMREMAGASFPRTPDVNNYVLTTYIDDPAPWETQSIGDDGHIKAGKIVDFIRYAREHDLNCAPQFVDPQMDRSNPDAQERSPGLRVVEKNEKGIVVNGVKAIGTGVAFADWIHIGTFFRPGIPGDQVIFAATPVNTPGVTIVCRESLVKDDKVEHPLAAQGDELDGMTVFENVFIPWSHVFHIGNPNHAKLYPQRVFDWLHYHALIRQMVRAELVAGLAVLITEHIGTNKIPAVQTRVAKLIGFHQAMLAHLIASEELGFHTPGGHYKPNILIYDFGRALYLENFSQMIYELVDLSGRSALIFASEDQWNDDKLNGWFERMNNGPVGRPHDRVKIGRVIRDLFLTDWGSRLFVFENFNGTPLQGIRMLTMQRAEFSGSGPYGKLARQVCGIDSAVTDDTEYRKTADYAKALDAARHQEEVALAGAMAI

SEQ ID NO: 29 - protein, 494 aa, Geobacillus sp.
MKMPAKTGKEYMERLKQAKSSVYIHGEKVEDVTVHPAFRNVVRSMAALYDRQYEKPEKMLYRSPTTGQPVGMTFIQPTTIDELIARREATQEWARMSAGMMGRSPDYLNAEVMAMGIANDLFAEDDPMFAENAKNYYEYAREHDISLTHTLIHPQMNRAKALHEQNDADVPLHLVERRKDGIIVSGIRLLATQGGITDEILVFPSTTKKSNTGEDPYALAFAIPNNTPGVKFICREAFDYGRSAWDHPLASRFEEGDAIVSFENVFVPWERVFVCGNSSICNRTFRETNAVVHMSHQVVAKNIVKTEFLLGVTLCLIEAIGIGEFQHVKDKGAEIMLVLETMKSHLYRAEHNAKRDRWGTMTPDFAALDAARNWYPRIYPRLAEIIRILGASGLMAIPTEADFQHEEIGDIVRRAMQGATVDGYERVQLFRLAWDLTMSAFGARQTHYEYYFFGDPVRMGMAYFDGYEKEPYKQFVREFLRGAKSVFIPADNKH

SEQ ID NO: 30 - protein, 494 aa, Geobacillus sp.
MKMPAKTGKEYMERLKQAKSSVYIHGEKVEDVTVHPAFRNVVRSMAALYDRQYEKPEKMLYRSPTTGQPVGMTFIQPTTIDELIARREATQEWARMSAGMMGRSPDYLNAEVMAMGIANDLFAEDDPMFAENAKNYYEYAREHDISLTHTLIHPQMNRAKALHEQNDADVPLHLVERRKDGIIVSGIRLLATQGGITDEILVFPSTTKKVMNGEDPYALAFAIPNNTPGVKFICREAFDYGRSAWDHPLASRFEEGDAIVSFENVFVPWERVFVCGNSSICNRTFRETNAVVHMSHQVVAKNIVKTEFLLGVTLCLIEAIGIGEFQHVKDKGAEIMLVLETMKSHLYRAEHNAKRDRWGTMTPDFAALDAARNWYPRIYPRLAEIIRILGASGLMAIPTEADFQHEEIGDIVRRAMQGATVDGYERVQLFRLAWDLTMSAFGARQTHYEYYFFGDPVRMGMAYFDGYEKEPYKQFVREFLRGAKSVFIPADNKH

SEQ ID NO: 31 - protein, 494 aa, Geobacillus sp.
MKMPAKTGKEYMERLKQAKSSVYIHGEKVEDVTVHPAFRNVVRSMAALYDRQYEKPEKMLYRSPTTGQPVGMTFIQPTTIDELIARREATQEWARMSAGMMGRSPDYLNAEVMAMGIANDLFAEDDPMFAENAKNYYEYAREHDISLTHTLIHPQMNRAKALHEQNDADVPLHLVERRKDGIIVSGIRLLATQGGITDEILVFPSTTKKATSAQNPYALAFAIPNNTPGVKFICREAFDYGRSAWDHPLASRFEEGDAIVSFENVFVPWERVFVCGNSSICNRTFRETNAVVHMSHQVVAKNIVKTEFLLGVTLCLIEAIGIGEFQHVKDKGAEIMLVLETMKSHLYRAEHNAKRDRWGTMTPDFAALDAARNWYPRIYPRLAEIIRILGASGLMAIPTEADFQHEEIGDIVRRAMQGATVDGYERVQLFRLAWDLTMSAFGARQTHYEYYFFGDPVRMGMAYFDGYEKEPYKQFVREFLRGAKSVFIPADNKH

SEQ ID NO: 32 - protein, 494 aa, Geobacillus sp.
MKMPAKTGKEYMERLKQAKSSVYIHGEKVEDVTVHPAFRNVVRSMAALYDRQYEKPEKMLYRSPTTGQPVGMTFIQPTTIDELIARREATQEWARMSAGMMGRSPDYLNAEVMAMGIANDLFAEDDPMFAENAKNYYEYAREHDISLTHTLLHPQMNRAKALHEQNDADVPLHLVERRKDGIIVSGIRLLATQGGITDEILVFPSTTKKATSGEDPYALAFAIPNNTPGVKFICREAFDYGRSAWDHPLASRFEEGDAIVSFENVFVPWERVFVCGNSSICNRTFRETNAVVHMSHQVVAKNIVKTEFLLGVTLCLIEAIGIGEFQHVKDKGAEIMLVLETMKSHLYRAEHNAKRDRWGTMTPDFAALDAARNWYPRIYPRLAEIIRILGASGLMAIPTEADFQHEEIGDIVRRAMQGATVDGYERVQLFRLAWDLTMSAFGARQTHYEYYFFGDPVRMGMAYFDGYEKEPYKQFVREFLRGAKSVFIPADNKH

SEQ ID NO: 33 - protein, 490 aa, Geobacillus sp.
AKTGKEYMERLKQAKSSVYIHGEKVEDVTVHPAFRNVVRSMAALYDRQYEKPEKMLYRSPTTGQPVGMTFIQPTTIDELIARREATQEWARMSAGMMGRSPDYLNAEVMAMGIANDLFAEDDPMFAENAKNYYEYAREHDISLTHTLIHPQMNRAKALHEQNDADVPLHLVERRKDGIIVSGIRLLATQGGITDEILVFPSTVKKATSGEDPYALAFAIPNNTPGVKFICREAFDYGRSAWDHPLASRFEEGDAIVSFENVFVPWERVFVCGNSSICNRTFRETNAVVHMSHQVVAKNIVKTEFLLGVTLCLIEAIGIGEFQHVKDKGAEIMLVLETMKSHLYRAEHNAKRDRWGTMTPDFAALDAARNWYPRIYPRLAEIIRILGASGLMAIPTEADFQHEEIGDIVRRAMQGATVDGYERVQLFRLAWDLTMSAFGARQTHYEYYFFGDPVRMGMAYFDGYEKEPYKQFVREFLRGAKSVFIPADNKH

SEQ ID NO: 34 - protein, 473 aa, Thermos thermophilus
ARTGAEYIEALKTRPPNLWYKGEKVEDPTTHPVFRGIVRTMAALYDLQHDPRYREVLTYEEEGKRHGMSFLIPKTKEDLKRRGQAYKLWADQNLGMMGRSPDYLNAVVMAYAASADYFGEFAENVRNYYRYLRDQDLATTHALTNPQVNRARPPSGQPDPYIPVGVVKQTEKGIVVRGARMTATFPLADEVLIFPSTLLQAGSEKYALAFALPTSTPGLHFVCREALVGGDSPFDHPLSSRVEEMDCLVIFDDVLVPWERVFILGNVELCNNAYAATGALNHMAHQVVALKTAKTEAFLGVAALMAEGIGADVYGHVQEKIAEIIVYLEAMRAFWTRAEEEAKENAYGLLVPDRGALDGARNLYPRLYPRIREILEQIGASGLITLPSEKDFKGPLGPFLEKFLQGAALEAKERVALFRLAWDMTLSGFGARQELYERFFFGDPVRMYQTLYNVYNKEPYKERIRAFLKESLK

SEQ ID NO: 35 - protein, 488 aa, Geobacillus sp.
MPAKTGKEYMERLKQAKSSVYIHGEKVEDVTVHPAFRNVVRSMAALYDRQYEKPEKMLYRSPTTGQPVGMTFIQPTTIDELIARREATQEWARMSAGMMGRSPDYLNAEVMAMGIANDLFAEDDPMFAENAKNYYEYAREHDISLTHTLIHPQMNRAKALHEQNDADVPLHLVERRKDGIIVSGIRLLATQGGITDEILVFPSTVKKATSGEDPYALAFAIPNNTPGVKFICREAFDYGRSAWDHPLASRFEEGDAIVSFENVFVPWERVFVCGNSSICNRTFRETNAVVHMSHQVVAKNIVKTEFLLGVTLCLIEAIGIGEFQHVKDKGAEIMLVLETMKSHLYRAEHNAKRDRWGTMTPDFAALDAARNWYPRIYPRLAEIIRILGASGLMAIPTEADFQHEEIGDIVRRAMQGATVDGYERVQLFRLAWDLTMSAFGARQTHYEYYFFGDPVRMGMAYFDGYEKEPYKQFVREFLRGAKSVFIPA

SEQ ID NO: 36 - protein, 491 aa, Streptomyces globisporus
TRPLTGEEYLESLRDAREVYLDGSRVKDVTAHPAFHNPARMTARLYDSLHDPAQKAVLTAPTDAGDGFTHRFFTAPRSVDDLVKDQAAIASWARKSYGWMGRSPDYKASFLGTLGANADFYEPFADNARRWYRESQEKVLYWNHAFLHPPVDRSEVGDVFIHVERETDAGLVVSGAKVVATGSALTHAAFISHWGLPIKDRKFALVATVPMDADGLKVICRPSYSANAATTGSPFDNPLSSRLDENDAILVLDQVLIPWENVFVYGNLGKVHLLAGQSGMIERATFHGCTRLAVKLEFIAGLLAKALDITGAKDFRGVQTRLGEVLAWRNLFWSLSDAAARNPVPWKNGTLLPNPQAGMAYRWFMQIGYPRVLEIVQQDVASGLMYVNSSTEDFRNPETGPYLEKYLRGSDGAGAVERVKVMKLLWDAVGSDFGGRHELYERNYSGNHENTRIELLLSQTASGKLDSYMDFAQACMDEYDLDGWTAPDLES

SEQ ID NO: 37 - protein, 491 aa, Geobacillus sp.
PAKTGKEYMERLKQAKSSVYIHGEKVEDVTVHPAFRNVVRSMAALYDRQYEKPEKMLYRSPTTGQPVGMTFIQPTTIDELIARREATQEWARMSAGMMGRSPDYLNAEVMAMGIANDLFAEDDPMFAENAKNYYEYAREHDISLTHTLIHPQMNRAKALHEQNDADVPLHLVERRKDGIIVSGIRLLATQGGITDEILVFPSTVKKATSGEDPYALAFAIPNNTPGVKFICREAFDYGRSAWDHPLASRFEEGDAIVSFENVFVPWERVFVCGNSSICNRTFRETNAVVHMSHQVVAKNIVKTEFLLGVTLCLIEAIGIGEFQHVKDKGAEIMLVLETMKSHLYRAEHNAKRDRWGTMTPDFAALDAARNWYPRIYPRLAEIIRILGASGLMAIPTEADFQHEEIGDIVRRAMQGATVDGYERVQLFRLAWDLTMSAFGARQTHYEYYFFGDPVRMGMAYFDGYEKEPYKQFVREFLRGAKSVFIPADNKH

SEQ ID NO: 38 - protein, 490 aa, Clostridium aminobutyricum; identical to SEQ ID NO: 5

SEQ ID NO: 39 - protein, 480 aa, Burkholderia cepacia
MRTGKQYLESLNDGRVVWVGNEKIDNVATHPLTRDYAERVAQFYDLHHRPDLQDVLTFVDADGVRRSRQWQDPKDAAGLRVKRKYHETILREIAAGSYGRLPDAHNYTFTTYADDPEVWEKQSIGAEGRNLTQNIHNFLKLLREKDLNCPLNFVDPQTDRSSDAAQARSPNLRIVEKTDDGIIVNGVKAVGTGIAFGDYMHIGCLYRPGIPGEQVIFAAIPTNTPGVTVFCRESTVKNDPAEHPLASQGDELDSTTVFDNVFIPWEQVFHIGNPEHAKLYPQRIFDWVHYHILIRQVLRAELIVGLAILITEHIGTSKLPTVSARVAKLVAFHLAMQAHLIASEETGFHTKGGRYKPNPLIYDFGRAHFLQNQMSVMYELLDLAGRSSLMIPSEGQWDDSQSGQWFVKLNNGPKGNPRERVQIGRVIRDLYLTDWGGRQFMFENFNGTPLFAVFAATMTRDDMSAAGTYGKFASQVCGIE

SEQ ID NO: 40 - protein, 469 aa, Cupriavidus necator
IRTGKQYLESLNDGRNVWVGNEKIDNVATHPKTRDYAQRHADFYDLHHRPDLQDVMTFVDKDGERRTMQWFGHYDKEQLRRKRKYHETIMREMAGASFPRTPDVNNYVLQTYIDDPSPWETQTIGAEGKVKAKNIVDFVNFAKKHDLNCAPQFVDPQMPGLRVIEKNDKGIVVSGVKAIGTGVAFADWIHIGVFFRPGIPGDQIIFAATPVNTPGVTIVCRESVVKEDPIEHPLASQGDELDGMTVFDNVFIPWSHVFHLGNPEHAKLYPQRVFDWLHYHALIRQSVRAELMAGLAILITEHIGTNKIPAVQTRVAKLIGFHQAMLAHIVASEELGFHTPGGAYKPNILIYDFGRALYLENFSQMIYELVDLSGRSALIFASEDQWNDEALNGWFERMNNGPVGQPHDRVKIGRVIRDLFLTDWGNRLFVFENFNGTPLQAIRMLTMQRAEFSAAGPYGTLARKVCGIE

SEQ ID NO: 41 - protein, 436 aa, Geobacillus sp.
TVHPAFRNVVRSMAALYDRQYEKPEKMLYRSPTTGQPVGMTFIQPTTIDELIARREATQEWARMSAGMMGRSPDYLNAEVMAMGIANDLFAEDDPMFAENAKNYYEYAREHDISLTHTLIHPQMNRAKALHEQNDADVPLHLVERRKDGIIVSGIRLLATQGGITDEILVFPSTVKKATSGEDPYALAFAIPNNTPGVKFICREAFDYGRSAWDHPLASRFEEGDAIVSFENVFVPWERVFVCGNSSICNRTFRETNAVVHMSHQVVAKNIVKTEFLLGVTLCLIEAIGIGEFQHVKDKGAEIMLVLETMKSHLYRAEHNAKRDRWGTMTPDFAALDAARNWYPRIYPRLAEIIRILGASGLMAIPTEADFQHEEIGDIVRRAMQGATVDGYERVQLFRLAWDLTMSAFGARQTHYEYYFFGDPVRMGMAYFDGYE

SEQ ID NO: 42 - protein, 377 aa, Oscillatoria sp.
AWNSQQIQFRKKVIQFAQQSLISDLIKNDKEEIFNRDAWQKCSEFGVHGWPIPARYGGQELDILTTAYALQGLGYGCKDNGLIFAMNAHIWACEMPLLTFGTEEQKEKYLPLLCRGGWIASHAATEPQAGSDIYSLKTTAQKDGDKYILNGYKHYVTNGTIADLFIIFATIDPSLGKEGLTTFMIEKDTPGLILSKPISKMGMRTAEVPELRLENCEVSAANRLGEEGTGLAIFNHSMEWERGFILAAAVGTMERLLEQSIRYARSHKQFGQAIGKFQLVANKLVEMKLRLENAKAYLYKVAWMKENKQMALLEASMANLYISEAWVQSCLEAIEIHGAYGYLTNTELERELRDAIASKFYSGTSEIQRVVIAKFLG

SEQ ID NO: 43 - protein, 470 aa, Geobacillus sp.
AKTGKEYMERLKQAKSSVYIHGEKVEDVTVHPAFRNVVRSMAALYDRQYEKPEKMLYRSPTTGQPVGMTFIQPTTIDELIARREATQEWARMSAGMMGRSPDYLNAEVMAMGIANDLFAEDDPMFAENAKNYYEYAREHDISLTHTLIHPQMNRAKALHEQNDADVPLHLVERRKDGIIVSGIRLLATQGGITDEILVFPSTVKKATSGEDPYALAFAIPNNTPGVKFICREAFDYGRSAWDHPLASRFEEGDAIVSFENVFVPWERVFVCGNSSICNRTFRETNAVVHMSHQVVAKNIVKTEFLLGVTLCLIEAIGIGEFQHVKDKGAEIMLVLETMKSHLYRAEHNAKRDRWGTMTPDFAALDAARNWYPRIYPRLAEIIRILGASGLMAIPTEADFQHEEIGDIVRRAMQGATVDGYERVQLFRLAWDLTMSAFGARQTHYEYYFFGDPVRMGMAYFDGYEKEPYKQ

SEQ ID NO: 44 - protein, 379 aa, Paraburkholderia phymatum
FFEARHRELAAGIEAWATQHLACVQHDDTDTTCRKLVRALGEAGWLKYGVGGAQYGGHGDTIDTRAVCLLRETLANHDGLADFALAMQGLGSGAITLAGTHEQKIRYLPRVSKGEAIAAFALSEPDAGSDVAAMSLQARAEGDCYVIDGDKTWISNGGIADFYVVFARTGEAPGARGISAFIVDADTPGLQIAERIDVIAPHPLARLHFDSARVPRSQMLGAPGEGFKIAMRTLDVFRTSVAAASLGFARRALQEGLARAASRKMFGQTLGDFQLTQTKLAQMALTIDSSALLVYRAAWLRDQGENVTREAAMAKWHASEGAQQVIDAAVQLWGGMGVQSGTTVERLYREIRALRIYEGATEVQQLIVGRDLLKAHAAQ

SEQ ID NO: 45 - protein, 472 aa, Ralstonia pickettii
MIRTGTQYLESLNDGRNVWVGNEKIDNVATHPKTRDYAQRHADFYDLHHRPDLQDVMTYIDEGGQRRAMQWFGHRDKEQLRRKRKYHETVMREMAGASFPRTPDVNNYVLTTYIDDPAPWETQSIGDDGHIKAGKIVDFIRYAREHDLNCAPQFVDPQERSPGLRVVEKNEKGIVVNGVKAIGTGVAFADWIHIGVFFRPGIPGDQVIFAATPVNTPGVTIVCRESLVKDDKVEHPLAAQGDELDGMTVFENVFIPWSHVFHIGNPNHAKLYPQRVFDWLHYHALIRQMVRAELVAGLAVLITEHIGTNKIPAVQTRVAKLIGFHQAMLAHLIASEELGFHTPGGHYKPNILIYDFGRALYLENFSQMIYELVDLSGRSALIFASEDQWNDDKLNGWFERMNNGPVGRPHDRVKIGRVIRDLFLTDWGSRLFVFENFNGTPLQGIRMLTMQRAEFSGSGPYGKLARQVCGID

(19) United States
(12) Patent Application Publication
(10) Pub. No.: US 2023/0340428 A1
(43) Pub. Date: Oct. 26, 2023

(54) POLYNUCLEOTIDE ENCODING AN AMINO ACID SEQUENCE, ENCODING AN OXIDOREDUCTASE

(71) Applicant: Evonik Operations GmbH, Essen (DE)

(72) Inventors: Steffen SCHAFFER, Herten (DE); Tobias BLATTERT, Essen (DE); Steffen OSSWALD, Nidderau (DE); Ronald MATHAE, Bielefeld (DE); Bo Roger SVENSSON, Lund (SE); Derek Thomas LOGAN, Veberod (SE); Rudy PANDJAITAN, Maison Alfort (FR); Matthieu NG FUK CHONG, Vanves (FR)

(73) Assignee: Evonik Operations GmbH, Essen (DE)

(21) Appl. No.: 18/005,086

(22) PCT Filed: Jul. 14, 2021
(86) PCT No.: PCT/EP2021/069597

§ 371 Date: Jan. 11, 2023

(30) Foreign Application Priority Data
Jul. 15, 2020 (EP) 20185930.3

(51) Int. Cl. C12N 9/02; C12P 13/22

(52) U.S. Cl. CPC C12N 9/0071; C12Y 114/14009; C12P 13/225

(57) ABSTRACT

A polynucleotide, encoding an amino acid sequence, encoding an oxidoreductase, that is ≥50% identical to an amino acid sequence of SEQ ID NO:1 (Geobacillus sp. PA9), SEQ ID NO:3 (Thermus thermophilus), SEQ ID NO:4 (Streptomyces globisporus), SEQ ID NO:5 (Clostridium aminobutyricum), SEQ ID:6 (Burkholderai cepacia), SEQ ID NO:8 (Oscillatoria sp. PCC 6506), or SEQ ID NO:9 (Paraburkholderia phymatum). The polynucleotide has an amino acid exchange in one or more of positions 202, 203, 204, 205, 206, 207, 208, 209, 210, 211, 212, 213, 214 of SEQ ID NO:1, or at a corresponding position of the amino acid sequence of SEQ ID NO:3, SEQ ID NO:4, SEQ ID:5, SEQ ID NO:6, SEQ ID NO:8, or SEQ ID NO:9.

The invention is in the field of fermentative processes for the production of fine chemicals and covers a polynucleotide encoding an amino acid sequence, encoding an oxidoreductase and a fermentative process using an oxidoreductase.

Patients suffering from Parkinson's disease experience symptoms like tremor, rigidity or bradykinesia, which are caused by low levels of dopamine due to cell death in the brain's basal ganglia. As dopamine is not able to pass the blood-brain-barrier, its precursor molecule L-DOPA (L-dihydroxyphenylalanine) is used as medication to reduce the patient's symptoms. Since its first launch in 1977 L-DOPA (Levodopa) advanced to be the first in line Parkinson treatment with a global market volume of 600 MT.

Existing production processes are based on chemical synthesis, specifically asymmetric hydrogenation or hydrogenation and chiral resolution. This is a well-established manufacturing process with poor conversion rate and overall efficiency. Alternatively, L-DOPA is produced via enzymatic conversion, namely enzymatic coupling of pyruvate and catechol. However, this enzymatic conversion process has the disadvantage of high raw material cost.

Fermentation processes using the 4-hydroxyphenylacetate 3-monooxygenase HpaB and the cognate 4-hydroxyphenylacetate 3-monooxygenase reductase HpaC from Escherichia coli (E. coli) are described in literature and can be advantageous due to higher conversion rate, enantioselectivity and a more economic process.

To achieve an economically attractive process, it is crucial to optimize tyrosine productivity on the one hand and conversion of L-tyrosine to L-DOPA by an increased HpaB-activity on the other hand.

A broad substrate spectrum of the 4-hydroxyphenylacetate monooxygenase enzyme was described in Prieto et al., 1993 and it was indicated, that the enzyme activity with the non-natural substrate L-tyrosine is only 5% of the enzyme activity with the natural substrate 4-HPA (4-hydroxyphenlyacetate). Thus, enzyme modelling was considered as a promising option to increase the enzyme activity.

Shen et al., Scientific Reports (2019) 9:7087/https://doi.org/10.1038/s41598-019-43577-w, p. 1-11, describes structural insights into catalytic versatility of the flavin-dependent hydroxylase (HpaB) from E. coli. Shen et al. refer that the 4-hydroxyphenylacetate 3-hydroxylase (EcHpaB) from E. coli is capable of efficient ortho-hydroxylation of a wide range of phenolic compounds and demonstrates great potential for broad chemoenzymatic applications. To understand the structural and mechanistic basis of its catalytic versatility, Shen et al. elucidated the crystal structure of EcHpaB by X-ray crystallography, which revealed a unique loop structure covering the active site. Shen et al. further performed mutagenesis studies of this loop to probe its role in substrate specificity and catalytic activity. Their results not only showed that the loop has great plasticity and strong tolerance towards extensive mutagenesis, but also suggested a flexible loop that enables the entrance and stable binding of substrates into the active site may be the key factor to the enzyme catalytic versatility. Loop sequences and structures of different EcHpaB mutants are reported. However, no enhanced selectivity for L-DOPA is described.

Fordjour et al., Microbial Cell Factories (2019) 18:74/https//doi.org/10.1186/s12934-019-1122-0, p. 1-10, describes metabolic engineering of E. coli BL21 (DE3) for de novo production of L-DOPA from D-glucose. E. coli BL21 (DE3) was engineered by deleting tyrR, ptsG, crr, pheA and pykF while directing carbon flow through the overexpression of galP and glk. TWA and ppsA were also overexpressed to enhance the accumulation of E4P and PEP. Site directed mutagenesis was applied on HpaB to optimize its activity. Three mutants, G883R, G883A, and L1231M, were identified to have improved activity as compared to the wild-type hpaB showing a 3.03-, 2.9- and 2.56-fold increase in L-DOPA production respectively. The use of strain LP-8 resulted in the production of 691.24 mg/L and 25.53 g/L of L-DOPA in shake flask and 5 L bioreactor, respectively.

EP3150712A1 (Symrise) describes biotechnological methods for providing 3,4-dihydroxypenyl compounds and methylated variants thereof. EP3150712A1 thereby relates to genetically modified enzymes obtained by rational design of the active site binding pocket of the prototypic enzyme 4-hydroxyphenylacetate 3-hydroxylase (4HPA3H) for hydroxylating a 4-hydroxyphenyl compound to yield a 3,4-dihydroxyphenyl compound and to biotechnological methods including in vivo and in vitro methods using said enzymes or catalytically active fragments thereof.

CN107541483A (Tianjin) describes a strain of E. coli for recombinant production of levodopa, its construction method and the application. CN107541483A thereby provides a method for producing L-DOPA with E. coli recombinant strain T002, wherein upregulation of the aroE gene is implemented to enhance the expression of 3-dehydrogenating enzyme shikimate dehydrogenase, which can in turn produce L-DOPA.

It was an object of the present invention to provide an economically attractive fermentative process for the production of L-DOPA with an optimized tyrosine production and a high conversion rate of L-tyrosine to L-DOPA. There is a need for a production process for L-DOPA having an improved yield or a higher end concentration of the product intracellularly and/or in the medium. This process needs to be scalable to ensure efficient production of L-DOPA.

A further object of the invention is to provide a cell which is modified in such a manner that it is capable of producing L-DOPA in high amounts.

The inventors of the present invention have surprisingly established that mutations of the oxidoreductase HpaB lead to an increase in the production of L-DOPA and a higher conversion rate of L-tyrosine to L-DOPA.

The invention relates to a polynucleotide, encoding an amino acid sequence, encoding an oxidoreductase, that is at least ≥50% identical to the amino acid sequence of SEQ ID NO:1 (Geobacillus sp. PA9), SEQ ID NO:3 (Thermus thermophilus), SEQ ID NO:4 (Streptomyces globisporus), SEQ ID NO:5 (Clostridium aminobutyricum), SEQ ID:6 (Burkholderai cepacia), SEQ ID NO:8 (Oscillatoria sp. PCC 6506), SEQ ID NO:9 (Paraburkholderia phymatum), characterized by an amino acid exchange in one or more of positions 202, 203, 204, 205, 206, 207, 208, 209, 210, 211, 212, 213, 214 of SEQ ID NO:1, or at a corresponding position of the amino acid sequence of SEQ ID NO:3, SEQ ID NO:4, SEQ ID:5, SEQ ID NO:6, SEQ ID NO:8, SEQ ID NO:9, wherein the amino acid exchange is not A210S or S212A.

According to the present invention an amino exchange at position 210 of SEQ ID NO:1 or at a corresponding position of the amino acid sequence of SEQ ID NO:3, SEQ ID NO:4, SEQ ID:5, SEQ ID NO:6, SEQ ID NO:8, SEQ ID NO:9, from alanine to serine and an amino exchange at position 212 of SEQ ID NO:1 or at a corresponding position of the amino acid sequence of SEQ ID NO:3, SEQ ID NO:4, SEQ ID:5, SEQ ID NO:6, SEQ ID NO:8, SEQ ID NO:9, from serine to alanine is excluded and shall not be part of the present invention.

In an alternative configuration, the polynucleotide has an amino acid exchange at one or more of positions 202, 203, 204, 205, 206, 207, 208, 209, 211, 213, 214 of SEQ ID NO:1, or at a corresponding position of the amino acid sequence of SEQ ID NO:3, SEQ ID NO:4, SEQ ID:5, SEQ ID NO:6, SEQ ID NO:8, SEQ ID NO:9.

In a preferred embodiment, the oxidoreductase is not the 4-hydroxyphenylacetate 3-monooxygenase of SEQ ID NO:2.

In a preferred embodiment, the amino acid exchange leads to an increase in the production of L-DOPA and/or a higher conversion rate of L-tyrosine to L-DOPA.

In a preferred embodiment, the oxidoreductase encoded by the nucleotide sequence is a 4-hydroxyphenylacetate 3-monooxygenase.

According to the present invention, the enzymes encoded by SEQ ID NO:1, SEQ ID NO:3, SEQ ID NO:4, SEQ ID:5, SEQ ID NO:6, SEQ ID NO:7, SEQ ID NO:8, SEQ ID NO:9 and SEQ ID NO:10 are 4-hydroxyphenylacetate 3-monooxygenases.

The homologue from Geobacillus sp. PA-9 was chosen as a starting point for rational enzyme design and a set of mutations was tested in a standardized whole-cell screening system. Transfer of promising results obtained in the screening system to a 1 L fermentation scale confirmed these results and showed significantly increased performance of the mutated enzymes in comparison to reference strains expressing the E. coli and Geobacillus sp. PA-9 wildtype genes.

The expression “a corresponding position of SEQ ID NO:1” or “a position comparable with position 202, 203, 204, 205, 206, 207, 208, 209, 210, 211, 213, 214 of the amino acid sequence” is taken to mean the fact that, by insertion or deletion of a codon encoding an amino acid in the N-terminal region (based on positions 202-214 of SEQ ID NO:1) of the encoded polypeptide, the positional statement and length statement in the case of an insertion is formally increased by one unit, or, in the case of a deletion, decreased by one unit. In the same manner, by insertion or deletion of a codon encoding an amino acid in the C-terminal region (based on positions 202-214) of the encoded polypeptide, the length statement, in the case of an insertion, is formally increased by one unit, or, in the case of a deletion, decreased by one unit. Such comparable positions may be readily identified by comparison of the amino acid sequences in the form of an “alignment”, for example using the Clustal W Programme (Thompson et al., Nucleic Acids Research 22, 4637-4680 (1994)) or the MAFFT Programme (Katoh et al., Genome Information 2005; 16(1),22-33). The expression “a corresponding position of SEQ ID NO:1”, when referring to different SEQ ID NO: from different species (such as SEQ ID NO:3-SEQ ID NO:10) refers to a homologous position within the crystal structure to be compared. Such a homologous position may also be identified by comparison of the amino acid sequences in the form of an “alignment” as described above or based on structural predictions.

In the present case, the positions 202, 203, 204, 205, 206, 207, 208, 209, 210, 211, 212, 213, 214 of SEQ ID NO:1 correspond to the following positions in the various sequences:
positions 194, 195, 196, 197, 198, 199, 200, 201, 202, 203, 204, 205 of SEQ ID NO:3;
positions 212, 213, 214, 215, 216, 217, 218, 219, 220, 221, 222 of SEQ ID NO:4;
positions 202, 203, 204, 205, 206, 207, 208, 209, 210 of SEQ ID NO:5;
positions 202, 203, 204, 205, 206, 207, 208, 209, 210, 211, 212 of SEQ ID NO:6;
positions 203, 204, 205, 206, 207, 208, 209, 210, 211, 212, 213 of SEQ ID NO:7;
positions 209, 210, 211, 212, 213, 214, 215, 216, 217 of SEQ ID NO:8;
positions 192, 193, 194, 195, 196, 197, 198, 199, 200, 201 of SEQ ID NO:9;
positions 203, 204, 205, 206, 207, 208, 209, 210, 211, 212, 213, 214, 215, 216, 217 of SEQ ID NO:10.

Regarding the oxidoreductases of the present invention, homologue HpaB enzyme sequences were aligned with Clustal Omega and in a second step, secondary structures of the HpaB sequences were predicted and the sequences were realigned using the secondary structures prediction.

Such insertions and deletions do not affect the enzymatic activity substantially. “Do not affect substantially” means that the enzymatic activity of said variants differs by a maximum of 10%, a maximum of 7.5%, a maximum of 5%, a maximum of 2.5%, or a maximum of 1%, from the activity of the polypeptide having the amino acid sequence of SEQ ID NO:1.

Preferably, the polynucleotide, encoding an amino acid sequence, encodes an oxidoreductase, that is at least ≥65%, identical to the amino acid sequence of SEQ ID NO:1 (Geobacillus sp. PA9), SEQ ID NO:3 (Thermus thermophilus), SEQ ID NO:4 (Streptomyces globisporus), SEQ ID NO:5 (Clostridium aminobutyricum), SEQ ID:6 (Burkholderai cepacia), SEQ ID NO:7 (Cupriavidus necator), SEQ ID NO:8 (Oscillatoria sp. PCC 6506), SEQ ID NO:9 (Paraburkholderia phymatum) characterized by an amino acid exchange in one or more of positions 202, 203, 204, 205, 206, 207, 208, 209, 210, 211, 212, 213, 214 of SEQ ID NO:1, or at a corresponding position of the amino acid sequence of SEQ ID NO:3, SEQ ID NO:4, SEQ ID:5, SEQ ID NO:6, SEQ ID NO:7, SEQ ID NO:8, SEQ ID NO:9.

More preferably, the polynucleotide, encoding an amino acid sequence, encodes an oxidoreductase, that is at least ≥90%, identical to the amino acid sequence of SEQ ID NO:1 (Geobacillus sp. PA9), SEQ ID NO:3 (Thermus thermophilus), SEQ ID NO:4 (Streptomyces globisporus), SEQ ID NO:5 (Clostridium aminobutyricum), SEQ ID:6 (Burkholderai cepacia), SEQ ID NO:7 (Cupriavidus necator), SEQ ID NO:8 (Oscillatoria sp. PCC 6506), SEQ ID NO:9 (Paraburkholderia phymatum), SEQ ID NO:10 (Ralstonia picketti),

characterized by an amino acid exchange in one or more of positions 202, 203, 204, 205, 206, 207, 208, 209, 210, 211, 212, 213, 214 of SEQ ID NO:1, or at a corresponding position of the amino acid sequence of SEQ ID NO:3, SEQ ID NO:4, SEQ ID:5, SEQ ID NO:6, SEQ ID NO:7, SEQ ID NO:8, SEQ ID NO:9, SEQ ID NO:10.

Preferably the polynucleotide encoding an amino acid sequence, encoding an oxidoreductase is selected from
SEQ ID NO:1 (Geobacillus sp. PA9) with an amino acid exchange at one or more of the positions 202, 203, 204, 205, 206, 207, 208, 209, 210, 211, 212, 213, 214;
SEQ ID NO:3 (Thermus thermophilus) with an amino acid exchange at one or more of the positions 194, 195, 196, 197, 198, 199, 200, 201, 202, 203, 204, 205; or SEQ ID NO:21;
SEQ ID NO:4 (Streptomyces globisporus) with an amino acid exchange at one or more of the positions 212, 213, 214, 215, 216, 217, 218, 219, 220, 221, 222; or SEQ ID NO:22;
SEQ ID NO:5 (Clostridium aminobutyricum) with an amino acid exchange at one or more of the positions 202, 203, 204, 205, 206, 207, 208, 209, 210; or SEQ ID NO:23;
SEQ ID:6 (Burkholderai cepacia) with an amino acid exchange at one or more of the positions 202, 203, 204, 205, 206, 207, 208, 209, 210, 211, 212; or SEQ ID NO:24;
SEQ ID NO:7 (Cupriavidus necator) with an amino acid exchange at one or more of the positions 203, 204, 205, 206, 207, 208, 209, 210, 211, 212, 213; or SEQ ID NO:25;
SEQ ID NO:8 (Oscillatoria sp. PCC 6506) with an amino acid exchange at one or more of the positions 209, 210, 211, 212, 213, 214, 215, 216, 217; or SEQ ID NO:26;
SEQ ID NO:9 (Paraburkholderia phymatum) with an amino acid exchange at one or more of the positions 192, 193, 194, 195, 196, 197, 198, 199, 200, 201; or SEQ ID NO:27;
SEQ ID NO:10 (Ralstonia picketti) with an amino acid exchange at one or more of the positions 203, 204, 205, 206, 207, 208, 209, 210, 211, 212, 213, 214, 215, 216, 217 or SEQ ID NO:28.

Those positions in the different sequences shall be meant by “a corresponding position of the amino acid sequence” according to the present invention.

The polynucleotide encoding an amino acid sequence, encodes an oxidoreductase, that is at least ≥90%, ≥92%, ≥94%, ≥96%, ≥97%, ≥98%, ≥99% or 100%, preferably ≥97%, particularly preferably ≥98%, very particularly preferably ≥99%, and extremely preferably 100%, identical to the amino acid sequence of SEQ ID NO:11, SEQ ID NO:12, SEQ ID NO:13 or SEQ ID NO:14 wherein SEQ ID NO:11, SEQ ID NO:12, SEQ ID NO:13 and SEQ ID NO:14, at position 207, or at a corresponding position of the amino acid sequence, has a proteinogenic amino acid other than L-valine.

The polynucleotide is preferably a sequence, wherein the polynucleotide is a replicable nucleotide sequence encoding the enzyme 4-hydroxyphenylacetate 3-monooxygenase from microorganisms of the genus Geobacillus, wherein the protein sequences encoded thereby contain a proteinogenic amino acid other than L-valine at the position corresponding to position 207 of SEQ ID NO:1.

In an advantageous configuration of the present invention the amino acid sequence encoded by the polynucleotide has, at the position 207 or a corresponding position, an amino acid which is selected from the group consisting of threonine, leucine, glutamine and glycine.

It is particularly preferred, when the protein sequences encoded by the polynucleotide
contain a proteinogenic amino acid other than L-threonine at position 206, or at a corresponding position of the amino acid sequence, preferably L-methionine or L-alanine; or
contain a proteinogenic amino acid other than L-lysine at position 208, or at a corresponding position of the amino acid sequence, preferably L-arginine.

In a preferred configuration the polynucleotide encoding an amino acid sequence, encodes an oxidoreductase that is at least ≥90%, ≥92%, ≥94%, ≥96%, ≥97%, ≥98%, ≥99% or ≥100%, preferably ≥97%, particularly preferably ≥98%, very particularly preferably ≥99%, and extremely preferably 100%, identical to the amino acid sequence of SEQ ID NO:15, SEQ ID NO:16, SEQ ID NO:17, SEQ ID NO:18, SEQ ID NO:19, SEQ ID NO:20, SEQ ID NO:29, SEQ ID NO:30, SEQ ID NO:31, SEQ ID NO:32.

The invention correspondingly also relates to polynucleotides and nucleic acid molecules comprising such sequences and encoding polypeptide variants of SEQ ID NO:1 to 20 and SEQ ID NO:29 to 32, which contain one or more insertion(s) or deletion(s). Preferably, the polypeptide contains a maximum of 5, a maximum of 4, a maximum of 3, or a maximum of 2, insertions or deletions of amino acids.

The invention further relates to a polypeptide comprising an amino acid sequence encoded by the nucleotide sequence according to the invention.

The invention preferably further relates to microorganisms of the genera Escherichia, Pseudomonas or Corynebacterium that comprise the polynucleotide, vectors and/or polypeptides according to the invention and in which microorganisms the nucleotide sequences encoding the 4-hydroxyphenylacetate monooxygenase enzyme are present preferably in overexpressed form.

Preferably the polypeptide encoded by the polynucleotide may show at the position 207 or at a corresponding position an amino acid which is selected from the group consisting of threonine, leucine, glutamine and glycine.

The protein sequences encoded thereby may contain a proteinogenic amino acid other than L-threonine at position 206, or at a corresponding position of the amino acid sequence, preferably L-methionine.

The protein sequences encoded thereby may further contain a proteinogenic amino acid other than L-lysine at position 208, or at a corresponding position of the amino acid sequence, preferably L-arginine.

The invention further relates to plasmids and vectors that comprise the polynucleotide according to the invention and optionally replicate in microorganisms of the genera Corynebacterium, Pseudomonas or Escherichia or are suitable therefor.

The invention further relates to microorganisms of the genera Corynebacterium, Pseudomonas or Escherichia that comprise the polynucleotide, vectors and polypeptides according to the invention.

The invention further relates to a microorganism according to the invention, characterized in that the polynucleotide according to the invention is integrated in a chromosome. Homologous recombination permits, with use of the vectors according to the invention, the exchange of DNA sections on the chromosome for polynucleotides according to the invention which are transported into the cell by the vector. For efficient recombination between the ring-type DNA molecule of the vector and the target DNA on the chromosome, the DNA region that is to be exchanged containing the polynucleotide according to the invention is provided at the ends with nucleotide sequences homologous to the target site; these determine the site of integration of the vector and of exchange of the DNA.

For instance, the polynucleotide according to the invention can be exchanged for the native hpaB gene at the native gene site in the chromosome or integrated at a further gene site.

The present invention provides a microorganism of the species E. coli, P. putida or C. glutamicum comprising any of the polynucleotides as claimed or any of the polypeptides as claimed or any of the vectors as claimed.

The microorganism may be a microorganism in which the polynucleotide is present in overexpressed form.

The microorganism may be characterized in that the microorganism has the capability of producing a fine chemical. The fine chemical being preferably L-dihydroxyphenylalanine (L-DOPA).

Overexpression is taken to mean, generally, an increase in the intracellular concentration or activity of a ribonucleic acid, a protein (polypeptide) or an enzyme, compared with the starting strain (parent strain) or wild-type strain, if this is the starting strain. A starting strain (parent strain) is taken to mean the strain on which the measure leading to the overexpression was carried out.

In the overexpression, the methods of recombinant overexpression are preferred. These include all methods in which a microorganism is produced using a DNA molecule provided in vitro. Such DNA molecules comprise, for example, promoters, expression cassettes, genes, alleles, encoding regions etc. These are converted into the desired microorganism by methods of transformation, conjugation, transduction or like methods.

The extent of the expression or overexpression can be established by measuring the amount of the mRNA transcribed by the gene, by determining the amount of the polypeptide, and by determining the enzyme activity.

Disclosed is a fermentative process for producing a fine chemical comprising the following steps:
a) fermentation of a microorganism comprising a polynucleotide encoding an amino acid sequence, encoding an oxidoreductase, that is at least ≥90%, ≥92%, ≥94%, ≥96%, ≥97%, ≥98%, ≥99% or 100%, preferably ≥97%, particularly preferably ≥98%, very particularly preferably ≥99%, and extremely preferably 100%, identical to the amino acid sequence of SEQ ID NO:1 or SEQ ID NO:3 to SEQ ID NO:32 in a medium,
b) accumulation of the fine chemical in the medium, wherein a fermentation broth is obtained.

The use of such a process according to the invention leads, as shown in Example 3, to an extraordinary increase in product concentration and L-DOPA:L-tyrosine ratio compared with the respective starting strain.

The culture medium or fermentation medium that is to be used must appropriately satisfy the demands of the respective strains. Descriptions of culture media of various microorganisms are contained in the handbook “Manual of Methods for General Bacteriology” of the American Society for Bacteriology (Washington D.C., USA, 1981). The terms culture medium and fermentation medium or medium are mutually exchangeable.

As carbon source, sugars and carbohydrates can be used, such as, e.g., glucose, sucrose, lactose, fructose, maltose, molasses, sucrose-containing solutions from beet sugar or sugar cane processing, starch, starch hydrolysate and cellulose, oils and fats, such as, for example, soybean oil, sunflower oil, groundnut oil and coconut fat, fatty acids, such as, for example, palmitic acid, stearic acid and linoleic acid, alcohols such as, for example, glycerol, methanol and ethanol, and organic acids, such as, for example, acetic acid or lactic acid.

As nitrogen source, organic nitrogen compounds such as peptones, yeast extract, meat extract, malt extract, corn-steep liquor, soybean meal and urea or inorganic compounds such as ammonium sulphate, ammonium chloride, ammonium phosphate, ammonium carbonate and ammonium nitrate can be used. The nitrogen sources can be used individually or as a mixture.

As phosphorus source, phosphoric acid, potassium dihydrogenphosphate or dipotassium hydrogenphosphate or the corresponding sodium-containing salts can be used.

The culture medium must, in addition, contain salts, for example in the form of chlorides or sulphates of metals such as, for example, sodium, potassium, magnesium, calcium and iron, such as, for example, magnesium sulphate or iron sulphate, which are necessary for growth. Finally, essential growth substances such as amino acids, for example homoserine and vitamins, for example thiamine, biotin or pantothenic acid, can be used in addition to the above-mentioned substances.

Said starting materials can be added to the culture in the form of a single batch or supplied in a suitable manner during the culturing.

Basic compounds such as sodium hydroxide, potassium hydroxide, ammonia or ammonia water, or acid compounds such as phosphoric acid or sulphuric acid, are used in a suitable manner for pH control of the culture. The pH is generally adjusted to 6.0 to 8.5, preferably 6.5 to 8. For control of foam development, antifoams can be used, such as, for example, polyglycol esters of fatty acids. For maintaining the stability of plasmids, suitable selectively acting substances such as, for example, antibiotics, can be added to the medium. The fermentation is preferably carried out under aerobic conditions. In order to maintain said aerobic conditions, oxygen or oxygen-containing gas mixtures such as, for example, air, are introduced into the culture. The use of liquids that are enriched with hydrogen peroxide is likewise possible. Optionally, the fermentation is carried out at superatmospheric pressure, for example at a superatmospheric pressure of 0.03 to 0.2 MPa. The temperature of the culture is usually 20° C. to 45° C., and preferably 25° C. to 40° C., particularly preferably 30° C. to 37° C. In the case of batch or fed-batch processes, the culturing is preferably continued until an amount sufficient for the measure of obtaining the desired organic chemical compound has formed. This goal is usually reached within 10 hours to 160 hours. In continuous processes, longer culture times are possible. Owing to the activity of the microorganisms, enrichment (accumulation) of the fine chemicals in the fermentation medium and/or in the cells of the microorganisms occurs.

Examples of suitable fermentation media may be found, inter alia, in patent documents U.S. Pat. Nos. 5,770,409, 5,990,350, 5,275,940, WO 2007/012078, U.S. Pat. No. 5,827,698, WO 2009/043803, U.S. Pat. No. 5,756,345 or U.S. Pat. No. 7,138,266; appropriate modifications may optionally be carried out to the requirements of the strains used.

The process may be characterized in that it is a process which is selected from the group consisting of batch process, fed-batch process, repetitive fed-batch process and continuous process.

The process may be further characterized in that the fine chemical or a liquid or solid fine chemical-containing product is obtained from the fine chemical-containing fermentation broth.

In a preferred configuration the fine chemical is L-dihydroxyphenylalanine (L-DOPA).

The performance of the processes or fermentation processes according to the invention with respect to one or more of the parameters selected from the group of concentration (compound formed per volume), yield (compound formed per carbon source consumed), volumetric productivity (compound formed per volume and time) and biomass-specific productivity (compound formed per cell dry mass or bio dry mass and time or compound formed per cell protein and time) or other process parameters and combinations thereof, is increased by at least 0.5%, at least 1%, at least 1.5% or at least 2%, based on processes or fermentation processes with microorganisms in which the promoter variant according to the invention is present.

Owing to the measures of the fermentation, a fermentation broth is obtained which contains the desired fine chemical, preferably amino acid or organic acid.

Then, a product in liquid or solid form that contains the fine chemical is provided or produced or obtained.

A fermentation broth is taken to mean, in a preferred embodiment, a fermentation medium or nutrient medium in which a microorganism was cultured for a certain time and at a certain temperature. The fermentation medium, or the media used during the fermentation, contains/contain all substances or components that ensure production of the desired compound and typically ensure growth and/or viability.

On completion of the fermentation, the resultant fermentation broth accordingly contains

a) the biomass (cell mass) of the microorganism resulting from growth of the cells of the microorganism,

b) the desired fine chemical formed in the course of the fermentation,

c) the organic by-products possibly formed in the course of the fermentation, and

d) the components of the fermentation medium used, or of the starting materials, that are not consumed by the fermentation, such as, for example, vitamins such as biotin, or salts such as magnesium sulphate.

The organic by-products include substances which are generated in addition to the respective desired compound by the microorganisms used in the fermentation and are possibly secreted.

The fermentation broth is withdrawn from the culture vessel or the fermentation container, optionally collected, and used for providing a product in liquid or solid form containing the fine chemical. The expression “obtaining the fine chemical-containing product” is also used therefor. In the simplest case, the fine chemical-containing fermentation broth withdrawn from the fermentation container is itself the product obtained.

By way of one or more of the measures selected from the group

a) partial (>0% to <80%) to complete (100%) or virtually complete (≥80%, ≥90%, ≥95%, ≥96%, ≥97%, ≥98%, ≥99%) removal of the water,

b) partial (>0% to <80%) to complete (100%) or virtually complete (≥80%, ≥90%, ≥95%, ≥96%, ≥97%, ≥98%, ≥99%) removal of the biomass, wherein this is optionally inactivated before the removal,

c) partial (>0% to <80%) to complete (100%) or virtually complete (≥80%, ≥90%, ≥95%, ≥96%, ≥97%, ≥98%, ≥99%, ≥99.3%, ≥99.7%) removal of the organic by-products formed in the course of the fermentation, and

d) partial (>0%) to complete (100%) or virtually complete (≥80%, ≥90%, ≥95%, ≥96%, ≥97%, ≥98%, ≥99%, ≥99.3%, ≥99.7%) removal of the components of the fermentation medium used or the starting materials that are not consumed by the fermentation, a concentration or purification of the desired organic chemical compound is achieved from the fermentation broth. In this manner, products are isolated that have a desired content of the compound.

The partial (>0% to <80%) to complete (100%) or virtually complete (≥80% to <100%) removal of the water (measure a)) is also termed drying.

In a variant of the process, by complete or virtually complete removal of the water, the biomass, the organic by-products and the non-consumed components of the fermentation medium used, pure (≥80% by weight, ≥90% by weight) or high-purity (≥95% by weight, ≥97% by weight, ≥99% by weight) product forms of the desired organic chemical compound, preferably amino acids, more preferably L-DOPA, are successfully arrived at. For the measures according to a), b), c) or d), a great variety of technical instructions are available in the prior art.

In the case of processes for producing L-DOPA, processes are preferred in which products are obtained that do not contain any components of the fermentation broth. These products are used, in particular, in human medicine, in the pharmaceuticals industry, and in the food industry.

The process according to the invention serves for the fermentative production of L-DOPA.

The invention finally relates to use of the microorganism according to the invention for the fermentative production of L-DOPA.

### Further Preferred Embodiments of the Present Invention are Summarized Below

The present invention is directed to a polynucleotide, encoding an amino acid sequence, encoding an oxidoreductase, that is at least ≥50% identical to the amino acid sequence of SEQ ID NO:1 (Geobacillus sp. PA9), SEQ ID NO:3 (Thermus thermophilus), SEQ ID NO:4 (Streptomyces globisporus), SEQ ID NO:5 (Clostridium aminobutyricum), SEQ ID:6 (Burkholderai cepacia), SEQ ID NO:8 (Oscillatoria sp. PCC 6506), SEQ ID NO:9 (Paraburkholderia phymatum),

characterized by an amino acid exchange in one or more of positions 202, 203, 204, 205, 206, 207, 208, 209, 210, 211, 212, 213, 214 of SEQ ID NO:1, or at a corresponding position of the amino acid sequence of SEQ ID NO:3, SEQ ID NO:4, SEQ ID:5, SEQ ID NO:6, SEQ ID NO:8, SEQ ID NO:9, wherein the amino acid exchange is not A210S or S212A.

In a preferred embodiment, the polynucleotide, encoding an amino acid sequence, encodes an oxidoreductase, that is at least ≥50% identical to the amino acid sequence of SEQ ID NO:1 (Geobacillus sp. PA9), SEQ ID NO:3 (Thermus thermophilus), SEQ ID NO:4 (Streptomyces globisporus), SEQ ID NO:5 (Clostridium aminobutyricum), SEQ ID:6 (Burkholderai cepacia), SEQ ID NO:8 (Oscillatoria sp. PCC 6506), SEQ ID NO:9 (Paraburkholderia phymatum),

characterized by an amino acid exchange in one or more of positions 202, 203, 204, 205, 206, 207, 208, 209, 210, 211, 212, 213, 214 of SEQ ID NO:1, or at positions 194, 195, 196, 197, 198, 199, 200, 201, 202, 203, 204, 205 of SEQ ID NO:3, or at positions 212, 213, 214, 215, 216, 217, 218, 219, 220, 221, 222 of SEQ ID NO:4, or at positions 202, 203, 204, 205, 206, 207, 208, 209, 210 of SEQ ID:5, or at positions 202, 203, 204, 205, 206, 207, 208, 209, 210, 211, 212 of SEQ ID NO:6, or at positions 209, 210, 211, 212, 213, 214, 215, 216, 217 of SEQ ID NO:8, or at positions 192, 193, 194, 195, 196, 197, 198, 199, 200, 201 of SEQ ID NO:9, wherein the amino acid exchange is not A210S or S212A.

In a further preferred embodiment, an amino exchange at positions 210 and 212 of SEQ ID NO:1 is excluded.

In a preferred embodiment, the polynucleotide, encoding an amino acid sequence, encodes an oxidoreductase, that is at least ≥65% identical to the amino acid sequence of SEQ ID NO:1 (Geobacillus sp. PA9), SEQ ID NO:3 (Thermus thermophilus), SEQ ID NO:4 (Streptomyces globisporus), SEQ ID NO:5 (Clostridium aminobutyricum), SEQ ID:6 (Burkholderai cepacia), SEQ ID NO:7 (Cupriavidus necator), SEQ ID NO:8 (Oscillatoria sp. PCC 6506), SEQ ID NO:9 (Paraburkholderia phymatum),

characterized by an amino acid exchange in one or more of positions 202, 203, 204, 205, 206, 207, 208, 209, 210, 211, 212, 213, 214 of SEQ ID NO:1, or at positions 194, 195, 196, 197, 198, 199, 200, 201, 202, 203, 204, 205 of SEQ ID NO:3, or at positions 212, 213, 214, 215, 216, 217, 218, 219, 220, 221, 222 of SEQ ID NO:4, or at positions 202, 203, 204, 205, 206, 207, 208, 209, 210 of SEQ ID:5, or at positions 202, 203, 204, 205, 206, 207, 208, 209, 210, 211, 212 of SEQ ID NO:6, or at positions 203, 204, 205, 206, 207, 208, 209, 210, 211, 212, 213 of SEQ ID NO:7, or at positions 209, 210, 211, 212, 213, 214, 215, 216, 217 of SEQ ID NO:8, or at positions 192, 193, 194, 195, 196, 197, 198, 199, 200, 201 of SEQ ID NO:9, wherein the amino acid exchange is not A210S or S212A.

In a further preferred embodiment, an amino exchange at positions 210 and 212 of SEQ ID NO:1 is excluded.

In a preferred embodiment, the polynucleotide, encoding an amino acid sequence, encodes an oxidoreductase, that is at least ≥90% identical to the amino acid sequence of SEQ ID NO:1 (Geobacillus sp. PA9), SEQ ID NO:3 (Thermus thermophilus), SEQ ID NO:4 (Streptomyces globisporus), SEQ ID NO:5 (Clostridium aminobutyricum), SEQ ID:6 (Burkholderai cepacia), SEQ ID NO:7 (Cupriavidus necator), SEQ ID NO:8 (Oscillatoria sp. PCC 6506), SEQ ID NO:9 (Paraburkholderia phymatum), SEQ ID NO:10 (Ralstonia pickettii),

characterized by an amino acid exchange in one or more of positions 202, 203, 204, 205, 206, 207, 208, 209, 210, 211, 212, 213, 214 of SEQ ID NO:1, or at positions 194, 195, 196, 197, 198, 199, 200, 201, 202, 203, 204, 205 of SEQ ID NO:3, or at positions 212, 213, 214, 215, 216, 217, 218, 219, 220, 221, 222 of SEQ ID NO:4, or at positions 202, 203, 204, 205, 206, 207, 208, 209, 210 of SEQ ID:5, or at positions 202, 203, 204, 205, 206, 207, 208, 209, 210, 211, 212 of SEQ ID NO:6, or at positions 203, 204, 205, 206, 207, 208, 209, 210, 211, 212, 213 of SEQ ID NO:7, or at positions 209, 210, 211, 212, 213, 214, 215, 216, 217 of SEQ ID NO:8, or at positions 192, 193, 194, 195, 196, 197, 198, 199, 200, 201 of SEQ ID NO:9 or at positions 203, 204, 205, 206, 207, 208, 209, 210, 211, 212, 213, 214, 215, 216, 217 of SEQ ID NO:10, wherein the amino acid exchange is not A210S or S212A.

In a further preferred embodiment, an amino exchange at positions 210 and 212 of SEQ ID NO:1 is excluded.

A preferred embodiment is directed to a polynucleotide, encoding an amino acid sequence encoding an oxidoreductase, that is at least ≥30%, identical to the amino acid sequence of,
SEQ ID NO:1 (Geobacillus sp. PA9) with an amino acid exchange at one or more of the positions 202, 203, 204, 205, 206, 207, 208, 209, 210, 211, 213, 214;
SEQ ID NO:3 (Thermus thermophilus) with an amino acid exchange at one or more of the positions 194, 195, 196, 197, 198, 199, 200, 201, 202, 203, 204, 205; or SEQ ID NO:21;
SEQ ID NO:4 (Streptomyces globisporus) with an amino acid exchange at one or more of the positions 212, 213, 214, 215, 216, 217, 218, 219, 220, 221, 222; or SEQ ID NO:22;
SEQ ID NO:5 (Clostridium aminobutyricum) with an amino acid exchange at one or more of the positions 202, 203, 204, 205, 206, 207, 208, 209, 210; or SEQ ID NO:23;
SEQ ID:6 (Burkholderai cepacia) with an amino acid exchange at one or more of the positions 202, 203, 204, 205, 206, 207, 208, 209, 210, 211, 212; or SEQ ID NO:24;
SEQ ID NO:7 (Cupriavidus necator) with an amino acid exchange at one or more of the positions 203, 204, 205, 206, 207, 208, 209, 210, 211, 212, 213; or SEQ ID NO:25;
SEQ ID NO:8 (Oscillatoria sp. PCC 6506) with an amino acid exchange at one or more of the positions 209, 210, 211, 212, 213, 214, 215, 216, 217; or SEQ ID NO:26;
SEQ ID NO:9 (Paraburkholderia phymatum) with an amino acid exchange at one or more of the positions 192, 193, 194, 195, 196, 197, 198, 199, 200, 201; or SEQ ID NO:27;
SEQ ID NO:10 (Ralstonia pickettii) with an amino acid exchange at one or more of the positions 203, 204, 205, 206, 207, 208, 209, 210, 211, 212, 213, 214, 215, 216, 217 or SEQ ID NO:28, wherein the amino acid exchange is not A210S or S212A.

A preferred embodiment is directed a polynucleotide, encoding an amino acid sequence encoding an oxidoreductase, that is at least ≥40% or at least ≥50%, or at least ≥60%, or at least ≥70%, or at least ≥80%, or at least ≥90%, or at least ≥95%, or at least ≥97%, or at least ≥98%, or at least ≥99% identical to the amino acid sequence of,
SEQ ID NO:1 (Geobacillus sp. PA9) with an amino acid exchange at one or more of the positions 202, 203, 204, 205, 206, 207, 208, 209, 210, 211, 212, 213, 214;
SEQ ID NO:3 (Thermus thermophilus) with an amino acid exchange at one or more of the positions 194, 195, 196, 197, 198, 199, 200, 201, 202, 203, 204, 205; or SEQ ID NO:21;
SEQ ID NO:4 (Streptomyces globisporus) with an amino acid exchange at one or more of the positions 212, 213, 214, 215, 216, 217, 218, 219, 220, 221, 222; or SEQ ID NO:22;
SEQ ID NO:5 (Clostridium aminobutyricum) with an amino acid exchange at one or more of the positions 202, 203, 204, 205, 206, 207, 208, 209, 210; or SEQ ID NO:23;
SEQ ID:6 (Burkholderai cepacia) with an amino acid exchange at one or more of the positions 202, 203, 204, 205, 206, 207, 208, 209, 210, 211, 212; or SEQ ID NO:24;
SEQ ID NO:7 (Cupriavidus necator) with an amino acid exchange at one or more of the positions 203, 204, 205, 206, 207, 208, 209, 210, 211, 212, 213; or SEQ ID NO:25;
SEQ ID NO:8 (Oscillatoria sp. PCC 6506) with an amino acid exchange at one or more of the positions 209, 210, 211, 212, 213, 214, 215, 216, 217; or SEQ ID NO:26;
SEQ ID NO:9 (Paraburkholderia phymatum) with an amino acid exchange at one or more of the positions 192, 193, 194, 195, 196, 197, 198, 199, 200, 201; or SEQ ID NO:27;
SEQ ID NO:10 (Ralstonia pickettii) with an amino acid exchange at one or more of the positions 203, 204, 205, 206, 207, 208, 209, 210, 211, 212, 213, 214, 215, 216, 217 or SEQ ID NO:28, wherein the amino acid exchange is not A210S or S212A.

A preferred embodiment is directed to a polynucleotide, encoding an amino acid sequence encoding an oxidoreductase, that is at least ≥30%, ≥40%, ≥50%, ≥60%, ≥70%, ≥80%, ≥90% identical to the amino acid sequence of SEQ ID NO:11, SEQ ID NO:12, SEQ ID NO:13 or SEQ ID NO:14 wherein SEQ ID NO:11, SEQ ID NO:12, SEQ ID NO:13 and SEQ ID NO:14, at position 207, or at a corresponding position of the amino acid sequence, has a proteinogenic amino acid other than L-valine.

A preferred embodiment is a polynucleotide, encoding an amino acid sequence encoding an oxidoreductase, that is at least ≥90%, ≥92%, ≥94%, ≥96%, ≥97%, ≥98%, ≥99% or ≥100%, preferably ≥97%, particularly preferably ≥98%, very particularly preferably ≥99%, and extremely preferably 100%, identical to the amino acid sequence of SEQ ID NO:11, SEQ ID NO:12, SEQ ID NO:13 or SEQ ID NO:14 wherein SEQ ID NO:11, SEQ ID NO:12, SEQ ID NO:13 and SEQ ID NO:14, at position 207, or at a corresponding position of the amino acid sequence, has a proteinogenic amino acid other than L-valine.

In a preferred embodiment the polynucleotide, encoding an amino acid sequence encodes an oxidoreductase, that is at least ≥30%, ≥40%, ≥50%, ≥60%, ≥70%, ≥80%, ≥90% identical to the amino acid sequence of SEQ ID NO:11, SEQ ID NO:12, SEQ ID NO:13 or SEQ ID NO:14, SEQ ID NO:15, SEQ ID NO:16, SEQ ID NO:17, SEQ ID NO:18, SEQ ID NO:19, SEQ ID NO:20, SEQ ID NO:29, SEQ ID NO:30, SEQ ID NO:31, SEQ ID NO:32.

In a preferred embodiment the polynucleotide, encoding an amino acid sequence encodes an oxidoreductase, that is at least ≥90%, ≥92%, ≥94%, ≥96%, ≥97%, ≥98%, ≥99% or ≥100%, preferably ≥97%, particularly preferably ≥98%, very particularly preferably ≥99%, and extremely preferably 100%, identical to the amino acid sequence of SEQ ID NO:11, SEQ ID NO:12, SEQ ID NO:13 or SEQ ID NO:14, SEQ ID NO:15, SEQ ID NO:16, SEQ ID NO:17, SEQ ID NO:18, SEQ ID NO:19, SEQ ID NO:20, SEQ ID NO:29, SEQ ID NO:30, SEQ ID NO:31, SEQ ID NO:32.

### EXAMPLES

The present invention will be described in more detail hereinafter with reference to exemplary embodiments.

### Example 1: Production of E. coli Strains with Different HpaB (Geobacillus) Mutants

A pOM17c-plasmid, which has been described previously in DE102004043748A1, where the complete sequence of the plasmid (containing the sequence of the cyanidase gene from Pseudomonas stutzeri AK61) is disclosed, was used as a starting point. Sequences of origin of replication and ampicillin resistance gene on this plasmid were maintained and the wildtype Geobacillus sp. PA-9 hpaB gene, the wildtype E. coli hpaC gene, the Pseudomonas oleovorans PalkB promoter and the E. coli rrnB terminator sequences, as well as the alkS transcriptional regulator gene (Yuste et al., J Bacteriol. 1998 October; 180(19):5218-26) were added. This plasmid was digested using the Geobacillus sp. wildtype single cutter restriction enzymes AgeI and PmlI [New England Biolabs GmbH, Brüningstraße 50, Geb. B852, 65926 Frankfurt am Main]. Synthesized DNA-fragments [Eurofins Genomics Germany GmbH Anzinger Str. 7a DE-85560 Ebersberg] were cloned with the plasmid backbone using the NEBuilder® HiFi DNA Assembly Master Mix [New England Biolabs GmbH Brüningstrasse 50, Geb. B852 65926 Frankfurt am Main] following the manufacturer's instructions. Thereby, the plasmid contained both E. coli hpaC and the different hpaB variants. After transformation of NEB® 5-alpha Electrocompetent E. coli [New England Biolabs GmbH Brüningstrasse 50, Geb. B852 65926 Frankfurt am Main], cells were plated on LB-Agar containing 100 μg/ml ampicillin. Restriction analysis and sequencing were done to select correctly cloned plasmids. If mentioned, the plasmid stabilizing toxin-antitoxin sequences hok/sok (Thisted, EMBO J. 1994 Apr. 15; 13(8):1950-9) and the cer determinant (Summers & Sherratt, 1984, DOI: 10.1016/0092-8674(84)90060-6) were added to the plasmids to increase plasmid stability.

L-tyrosine producing strain DPD6021-S was used for the transformation of the selected plasmids. DPD6021-S is an S-phase-variant of DPD4145, which is described in U.S. Pat. No. 7,700,328 B2 and where additionally the pMT100 plasmid was eliminated. For plasmid transformation, the strain was inoculated to an OD₆₀₀ of 0.05 from an LB-overnight culture and grown to an OD₆₀₀ of 0.7. After 30 min incubation on ice, the cells were harvested (10 min, 5500 g; 4° C.) and washed twice with 50 ml H₂O_{demin}. After an additional wash step with 1 ml pre-chilled 10% (v/v) Glycerol, cells were resuspended in 200 μl pre-chilled 10% (v/v) glycerol and aliquots of 40 μl were used for transformation. Therefore, 100 ng of plasmid were transferred to an electroporation cuvette, mixed with 40 μl cell solution and pulsed in a Gene Pulser Xcell Electorporation System [Bio-Rad Laboratories GmbH, Kapellenstraße 12, D-85622 Feldkirchen] at 2500 V, 200Ω & 25 μF. After the addition of 1 ml SOC media and regeneration for 45 min at 37° C., 100 μl of the suspension were plated to LB-Agar containing 100 μg/ml of Ampicillin. The plasmid was isolated from resistant colonies and authenticity of the plasmids was confirmed by restriction analysis.

The following strains were generated:
hpaBC_Ec: E. coli hpaB (wildtype; reference);
hpaB_Gs:
hpaB_V207L_Gs:
hpaB_V207T_Gs:
hpaB_V207Q_Gs:
hpaB_V207G_Gs:
hpaB_V207T_K208R_Gs:
hpaB_T206M_V207T_Gs:
hpaB_T206A_V207T_Gs:
Geobacillus sp. PA-9 hpaB (wildtype);
Geobacillus sp. PA-9 hpaB (Mutation V207L);
Geobacillus sp. PA-9 hpaB (Mutation V207T);
Geobacillus sp. PA-9 hpaB (Mutation V207Q);
Geobacillus sp. PA-9 hpaB (Mutation V207G);
Geobacillus sp. PA-9 hpaB (Mutation V207T_K208R);
Geobacillus sp. PA-9 hpaB (Mutation T206M_V207T);
Geobacillus sp. PA-9 hpaB (Mutation T206A_V207T).
hpaB_V207T_A210S_T211N_S212T: Geobacillus sp. PA-9 hpaB (Mutation V207T_A210S_T211N_S212T)
hpaB_V207T_A210V_T211M_S212N: Geobacillus sp. PA-9 hpaB (Mutation V207T_A210V_T211M_S212N)
hpaB_V207T_G213A_E214Q_D215N: Geobacillus sp. PA-9 hpaB (Mutation V207T_G213A_E214Q_D215N)
hpaB_1152 L_V207T: Geobacillus sp. PA-9 hpaB (Mutation 1152L_V207T)

### Example 2: Production of L-DOPA and L-Tyrosine (Using BioLector Screening)

DPD6021-S strains with plasmids expressing wildtype or mutated variants of the Geobacillus sp. PA-9 gene as well as a reference strain expressing the wildtype E. coli gene were tested in a 20 BioLector [m2p-labs; Arnold-Sommerfeld-Ring 2, 52499 Baesweiler] small scale test system.

Therefore, the strains were cultivated in 10 ml LB-media (100 μg/ml ampicillin) in baffled shake flasks at 37° C., 200 rpm for 18 h. Cultures were seeded into BioLector Flowerplates containing 1 ml LB-media, pH 5.5 (supplemented with 7.5 mM L-tyrosine; 100 μg/ml ampicillin; 0.25% (v/v) DCPK) to yield a starting OD₆₀₀ of 0.1. Cultivation was done at 37° C., 1200 rpm and relative humidity of 85% for 24 h, until the process was stopped, and L-DOPA and L-tyrosine concentrations were measured using High performance liquid chromatography (HPLC).

HPLC was performed on an Agilent 1200 (Agilent Technologies, Palo Alto, Calif.). An Inertsil ODS-3, 5 μm, 4.6×150 mm column (Agilent Technologies) was used. The method used required a column flow rate of 1.00 ml/min with a stop time of 18 minutes. The mobile phase was composed of ratios of Solvent A (2.72 g/L KH2PO4, 2.5 ml/L concentrated phosphoric acid, 40 ml/L acetonitrile) and Solvent B (acetonitrile) as described in table 1.

TABLE 1
Composition of mobile phase for HPLC analysis

| Time [min] | Solvent A [%] | Solvent B [%] |
|---|---|---|
| 0 | 100 | 0 |
| 6.5 | 100 | 0 |
| 10 | 75 | 25 |
| 13 | 75 | 25 |
| 13.01 | 100 | 0 |
| 18 | 100 | 0 |

The spectrum was scanned from 100 nm to 380 nm, with signal for L-DOPA being recorded at 290 nm and a retention time of 2.8 min, signal for L-tyrosine being recorded at 278 nm and a retention time of 3.9 minutes.

TABLE 2
Production of L-DOPA and L-tyrosine with
single mutants using BioLector screening

| | L-DOPA [mM] | L-Tyrosin [mM] |
|---|---|---|
| LD-EC-12 [hpaBC_Ec] | 2.7 | 5.4 |
| hpaB_Gs | 3.6 | 4.5 |
| hpaB_V207Q_Gs | 4.0 | 4.0 |
| hpaB_V207T_Gs | 4.3 | 3.6 |
| hpaB_V207L_Gs | 4.4 | 3.8 |

TABLE 3
Production of L-DOPA and L-tyrosine with single
and multiple mutants using BioLector screening
(plasmid containing plasmid stabilizing elements)

| | L-DOPA | L-Tyrosin |
|---|---|---|
| | [mM] | [mM] |
| hpaB_V207T_Gs | 5.2 | 3.5 |
| hpaB_T206M_V207T_Gs | 6.4 | 2.2 |
| hpaB_V207T_K208R_Gs | 5.4 | 3.6 |
| hpaB_V207G_Gs | 4.4 | 4.4 |
| hpaB_T206A_V207T_Gs | 4.7 | 3.8 |
| hpaB_V207T_A210S_T211N_S212T | 4.0 | 4.5 |
| hpaB_V207T_A210V_T211M_S212N | 4.0 | 4.6 |
| hpaB_V207T_G213A_E214Q_D215N | 3.8 | 4.7 |
| hpaB_I152L_V207T | 4.0 | 5.0 |

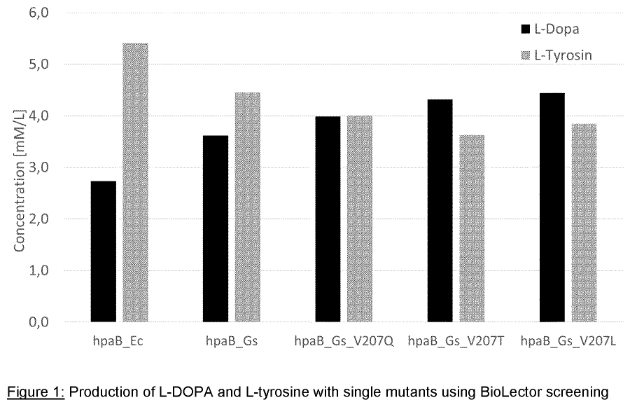

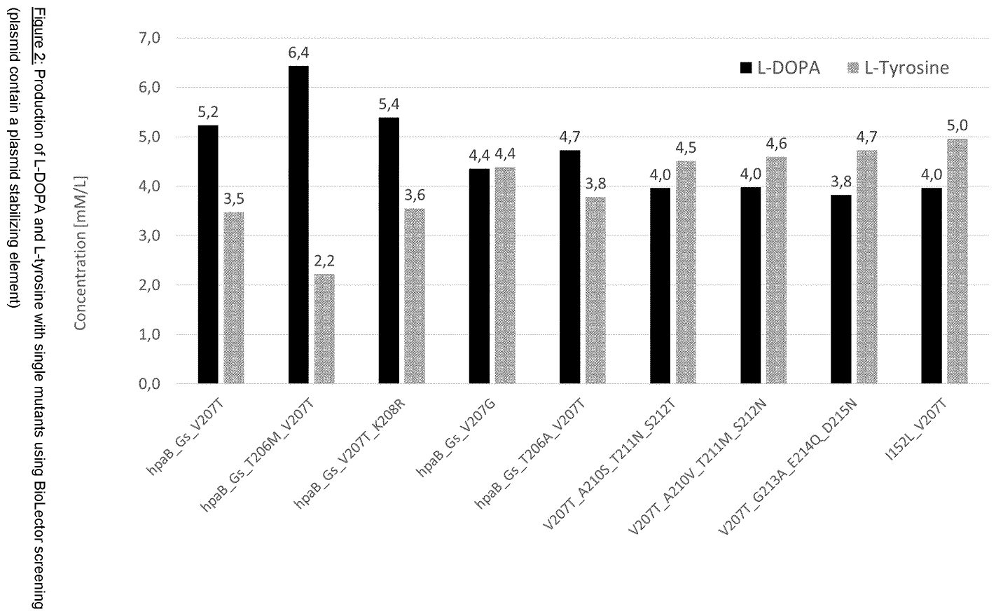

The results of the screening are summarized in table 2 and table 3 and visualized in FIG. 1 and FIG. 2. FIG. 1 shows the production of L-DOPA and L-tyrosine with single mutants using BioLector screening and FIG. 2 shows the production of L-DOPA and L-tyrosine with single and multiple mutants using BioLector screening (plasmid contain a plasmid stabilizing element).

### Example 3: Production of L-DOPA and L-Tyrosine (Fermentative Process)

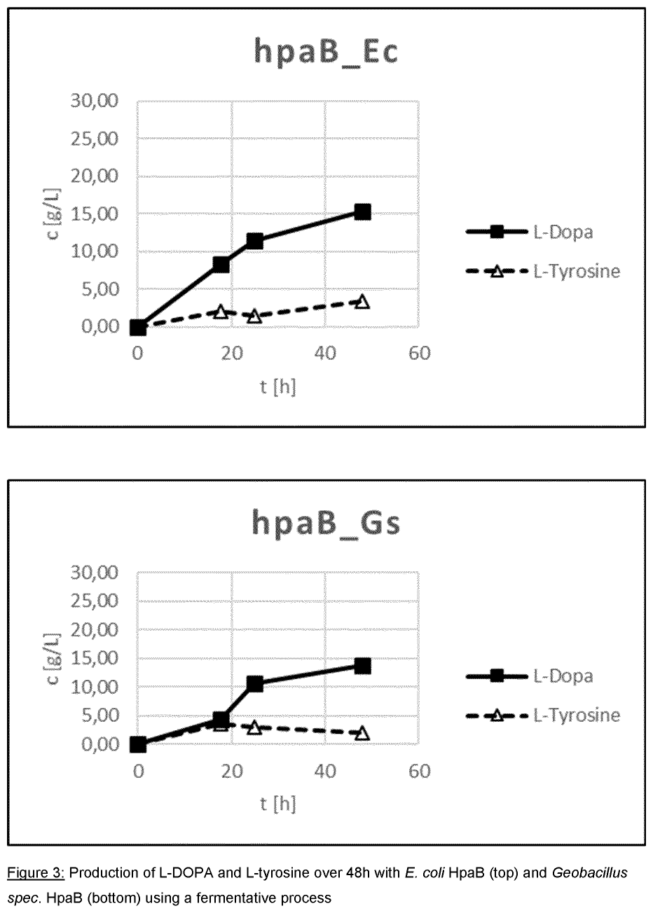

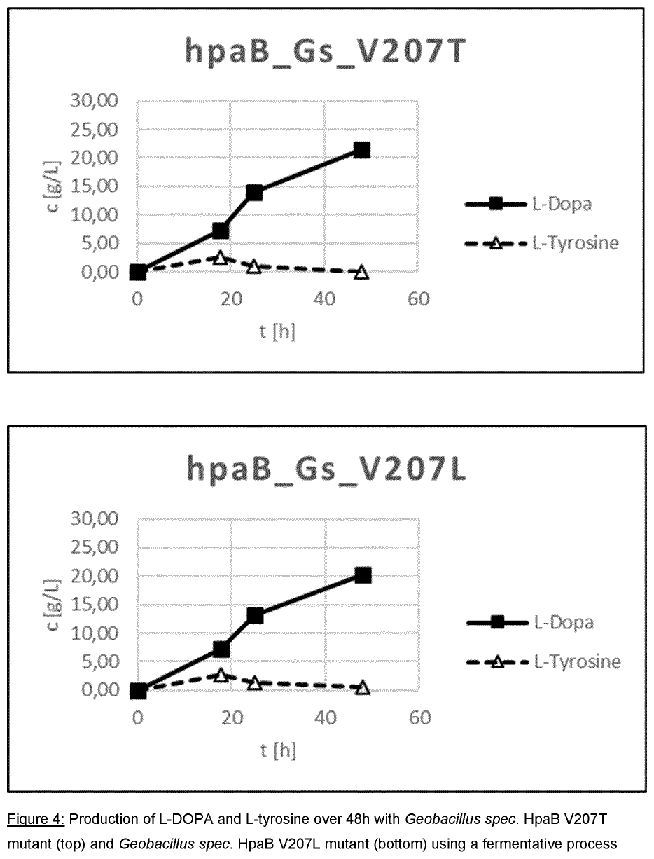

Fermentation was carried out as described in Example 8 of U.S. Pat. No. 7,700,328 B2 for strain DPD4145 and the strains were evaluated for production of L-tyrosine and L-DOPA. Unlike in Example 8 of U.S. Pat. No. 7,700,328 B2, fermentation was not induced with IPTG, but with Dicyclopropyl ketone (DCPK) and fermentation was performed in the presence of ampicillin (100 mg/L) at pH 6.8. Samples were drawn from the fermenter periodically and analyzed for L-tyrosine, L-DOPA and biomass concentration. The results are summarized in tables 4-6, table 4 showing the production of L-DOPA over 48 h, table 5 showing the production of L-tyrosine over 48 h for the same fermentation process and table 6 showing the ratio of L-DOPA/L-tyrosine. The results are visualized in corresponding FIGS. 3-4.

FIG. 3 shows the production of L-DOPA and L-tyrosine over 48 h with E. coli HpaB (top) and Geobacillus spec. HpaB (bottom) using a fermentative process and FIG. 4 shows the production of L-DOPA and L-tyrosine over 48 h with Geobacillus spec. HpaB V207T mutant (top) and Geobacillus spec. HpaB V207L mutant (bottom) using a fermentative process.

TABLE 4
Production of L-DOPA with fermentative process

| | L-DOPA [g/L] | | | |
|---|---|---|---|---|
| t [h] | hpaB_Ec | hpaB_Gs | hpaB_Gs_V207T | hpaB_Gs_V207L |
| 0 | 0.0 | 0.0 | 0.0 | 0.0 |
| 18 | 8.2 | 4.3 | 7.2 | 7.3 |
| 25 | 11.4 | 10.6 | 13.9 | 13.1 |
| 48 | 15.3 | 13.7 | 21.4 | 20.3 |

TABLE 5
Production of L-Tyrosine with fermentative process

| | L-Tyrosine [g/L] | | | |
|---|---|---|---|---|
| t [h] | hpaB_Ec | hpaB_Gs | hpaB_Gs_V207T | hpaB_Gs_V207L |
| 0 | 0.0 | 0.0 | 0.0 | 0.0 |
| 18 | 2.0 | 3.6 | 2.5 | 2.7 |
| 25 | 1.4 | 3.0 | 1.1 | 1.3 |
| 48 | 3.4 | 1.9 | below detection limit | 0.5 |

TABLE 6
Ratio of L-DOPA/L-Tyrosine with fermentative process

| | Ratio L-D/L-T | | | |
|---|---|---|---|---|
| t [h] | hpaB_Ec | hpaB_Gs | hpaB_Gs_V207T | hpaB_Gs_V207L |
| 0 | | | | |
| 18 | 4.0 | 1.2 | 2.9 | 2.7 |
| 25 | 8.2 | 3.5 | 12.9 | 9.7 |
| 48 | 4.5 | 7.1 | L-tyrosine below | 42.5 |
| | | | detection limit | |

It could be clearly shown that by using HpaB from Geobacillus as a wildtype enzyme and with different mutations for the fermentative production of L-DOPA for 48 h, the ratio of L-DOPA/L-tyrosine was higher as when using the E. coli wildtype enzyme (table 6). The mutants of the Geobacillus HpaB enzyme even have higher values for the ratio of L-DOPA/L-tyrosine.

Further mutants Gs_V207T-T206M and Gs_V207T-K208R were also tested in a fermentative process and similar results were obtained with high values for the ratio of L-DOPA/L-tyrosine.

### Example 5: Analysis of Structural Homologies

Regarding the oxidoreductases of the present invention, the Geobacillus sp. HpaB sequence was used to search the Protein Data Bank (PDB) for structural homologues. The structural alignment was constructed using MATT (Menke, M., Berger, B. & Cowen, L. Matt: Local Flexibility Aids Protein Multiple Structure Alignment. PLoS Comput. Biol. 4, el 0 (2008)).

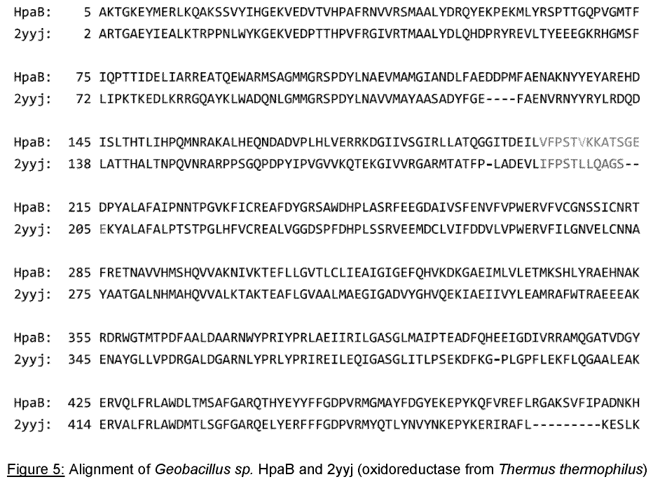

Such an alignment for the oxidoreductases of the present invention is shown in FIG. 5, which shows an alignment of Geobacillus sp. HpaB and 2yyj (oxidoreductase from Thermus thermophilus).

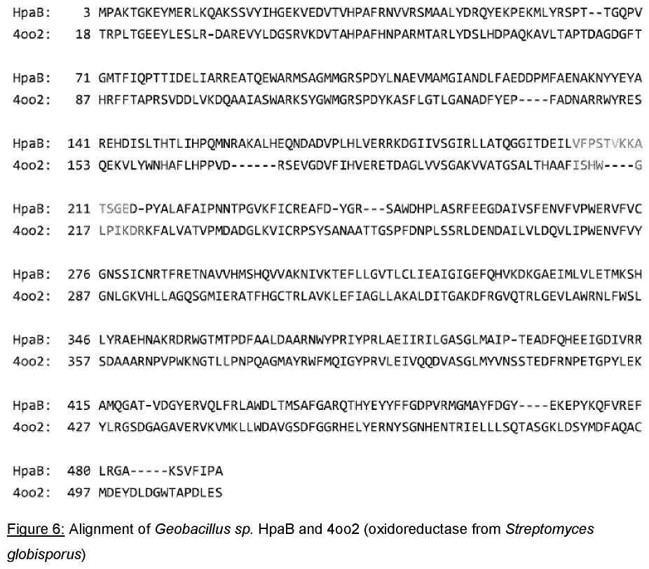

FIG. 6: Alignment of Geobacillus sp. HpaB and 4002 (oxidoreductase from Streptomyces globisporus)

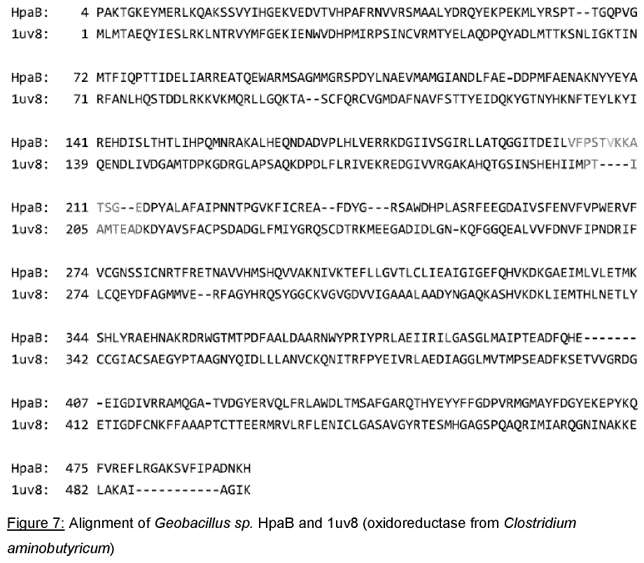

FIG. 7: Alignment of Geobacillus sp. HpaB and 1 uv8 (oxidoreductase from Clostridium aminobutyricum)

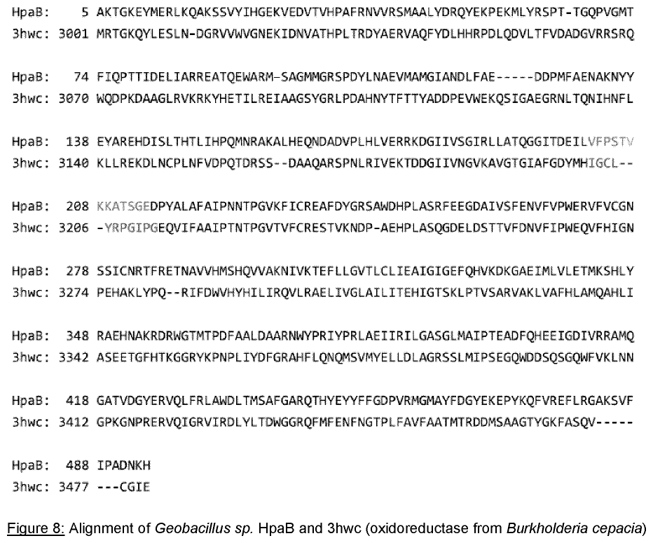

FIG. 8: Alignment of Geobacillus sp. HpaB and 3hwc (oxidoreductase from Burkholderia cepacia)

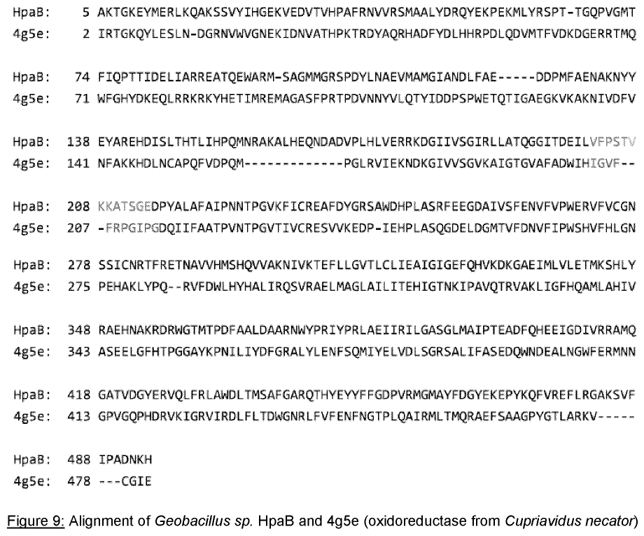

FIG. 9: Alignment of Geobacillus sp. HpaB and 4g5e (oxidoreductase from Cupriavidus necator)

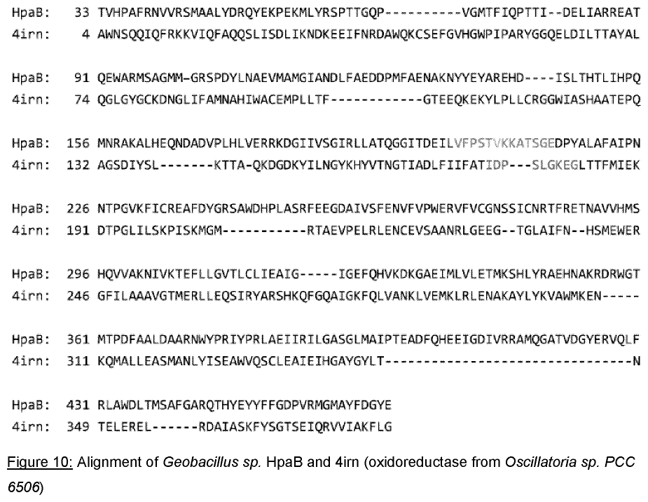

FIG. 10: Alignment of Geobacillus sp. HpaB and 4irn (oxidoreductase from Oscillatoria sp. PCC 6506)

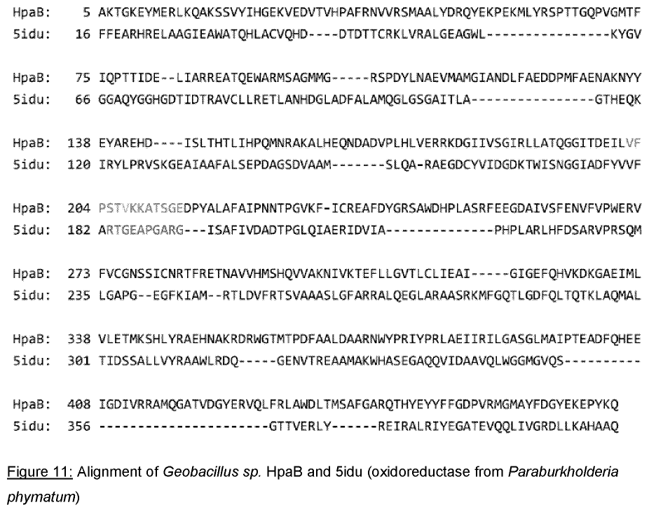

FIG. 11: Alignment of Geobacillus sp. HpaB and 5idu (oxidoreductase from Paraburkholderia phymatum)

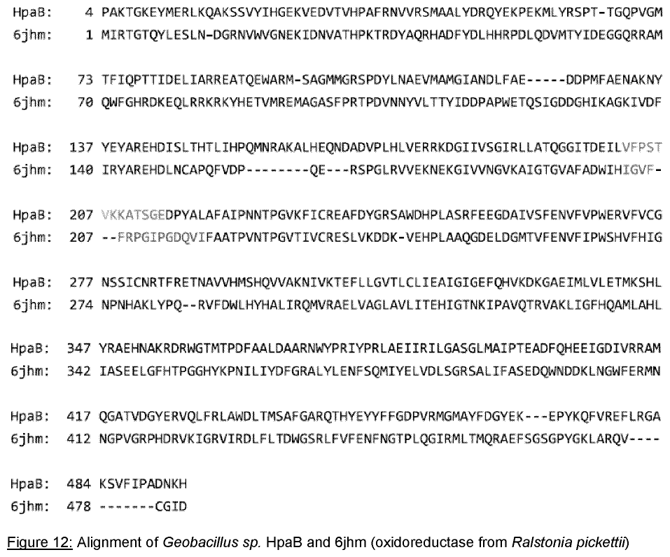

FIG. 12: Alignment of Geobacillus sp. HpaB and 6jhm (oxidoreductase from Ralstonia pickettii)

The superposition of the modeled HpaB structures shows that the proteins are well aligned.

### Example 6: Production of E. coli Strains with Different Oxidoreductase Mutants

Restriction of the pOM17c plasmid described in example 1 bearing the wildtype Geobacillus sp. PA-9 hpaB gene and the wildtype E. coli hpaC gene with the Enyzme Earl [New England Biolabs GmbH Brüningstrasse 50, Geb. B852 65926 Frankfurt am Main] is done to remove the wildtype Geobacillus sp. PA-9 hpaB gene, as well as the PalkB, alkS sequences and a part of the E. coli hpaC sequence. The resulting plasmid backbone is cloned with synthesized DNA fragments containing the previously removed PalkB and alkS sequences and a part of the E. coli hpaC sequence as well as the wildtype sequences of genes coding for the enzymes with amino acid sequences of SEQ ID NO:3 (Thermus thermophilus), SEQ ID NO:4 (Streptomyces globisporus), SEQ ID NO:5 (Clostridium aminobutyricum), SEQ ID:6 (Burkholderai cepacian), SEQ ID NO:7 (Cupriavidus necator), SEQ ID NO:8 (Oscillatoria sp. PCC 6506), SEQ ID NO:9 (Paraburkholderia phymatum), SEQ ID NO:10 (Ralstonia pickettii).

Furthermore, the mentioned plasmid backbone is cloned with synthesized DNA fragments containing the previously removed PalkB and alkS sequences and a part of the E. coli hpaC sequence as well as mutant sequences of genes resulting in enzyme variants with improved activity with the amino acid sequences of SEQ ID NO:21, SEQ ID NO:22, SEQ ID NO:23, SEQ ID NO:24, SEQ ID NO:25, SEQ ID NO:26, SEQ ID NO:27, SEQ ID NO:28.

The resulting plasmids are used for transformation of the L-tyrosine producing strain DPD6021-S as described in Example 1.

The resulting strains are cultivated in small scale screening system as described in Example 2 or fermentation as described in Example 3 to evaluate the conversion rate of the strains expressing mutant enzyme variants. L-DOPA formation is quantified as described in Example 1. L-DOPA production was detected for all variants.

Protein sequences

| | |
|---|---|
| SEQ ID NO: 1 | Geobacillus sp. PA9 HpaB |
| SEQ ID NO: 2 | E. coli (HpaB) 4-hydroxyphenylacetate |
| | 3-monooxygenase oxygenase component |
| SEQ ID NO: 3 | Thermus thermophilus |
| SEQ ID NO: 4 | Streptomyces globisporus |
| SEQ ID NO: 5 | Clostridium aminobutyricum |
| SEQ ID NO: 6 | Burkholderia cepacia |
| SEQ ID NO: 7 | Cupriavidus necator |
| SEQ ID NO: 8 | Oscillatoria sp. PCC 6506 |
| SEQ ID NO: 9 | Paraburkholderia phymatum |
| SEQ ID NO: 10 | Ralstonia pickettii |
| SEQ ID NO: 11 | Geobacillus sp. PA9 HpaB V207L |
| SEQ ID NO: 12 | Geobacillus sp. PA9 HpaB V207T |
| SEQ ID NO: 13 | Geobacillus sp. PA9 HpaB V207Q |
| SEQ ID NO: 14 | Geobacillus sp. PA9 HpaB V207G |
| SEQ ID NO: 15 | Geobacillus sp. PA9 HpaB T206M |
| SEQ ID NO: 16 | Geobacillus sp. PA9 HpaB T206A |
| SEQ ID NO: 17 | Geobacillus sp. PA9 HpaB K208R |
| SEQ ID NO: 18 | Geobacillus sp. PA9 HpaB V207T_K208R |
| SEQ ID NO: 19 | Geobacillus sp. PA9 HpaB T206M_V207T |
| SEQ ID NO: 20 | Geobacillus sp. PA9 HpaB T206A_V207T |
| SEQ ID NO: 21 | Thermus thermophilus (mutated) |
| SEQ ID NO: 22 | Streptomyces globisporus (mutated) |
| SEQ ID NO: 23 | Clostridium aminobutyricum (mutated) |
| SEQ ID NO: 24 | Burkholderia cepacia (mutated) |
| SEQ ID NO: 25 | Cupriavidus necator (mutated) |
| SEQ ID NO: 26 | Oscillatoria sp. PCC 6506 (mutated) |
| SEQ ID NO: 27 | Paraburkholderia phymatum (mutated) |
| SEQ ID NO: 28 | Ralstonia pickettii (mutated) |
| SEQ ID NO: 29 | Geobacillus sp. PA9 HpaB |
| | V207T_A210S_T211N_S212T |
| SEQ ID NO: 30 | Geobacillus sp. PA9 HpaB |
| | V207T_A210V_T211M_S212N |
| SEQ ID NO: 31 | Geobacillus sp. PA9 HpaB |
| | V207T_G213A_E214Q_D215N |
| SEQ ID NO: 32 | Geobacillus sp. PA9 HpaB I152L_V207T |

1. A polynucleotide, encoding an amino acid sequence, encoding an oxidoreductase, that is ≥50% identical to an amino acid sequence of SEQ ID NO:1 (Geobacillus sp. PA9), SEQ ID NO:3 (Thermus thermophilus), SEQ ID NO:4 (Streptomyces globisporus), SEQ ID NO:5 (Clostridium aminobutyricum), SEQ ID:6 (Burkholderai cepacia), SEQ ID NO:8 (Oscillatoria sp. PCC 6506), or SEQ ID NO:9 (Paraburkholderia phymatum),
wherein the polynucleotide has an amino acid exchange in one or more of positions 202, 203, 204, 205, 206, 207, 208, 209, 210, 211, 212, 213, 214 of SEQ ID NO:1, or at a corresponding position of the amino acid sequence of SEQ ID NO:3, SEQ ID NO:4, SEQ ID:5, SEQ ID NO:6, SEQ ID NO:8, or SEQ ID NO:9,
wherein the amino acid exchange is not A210S or S212A.

2. The polynucleotide, encoding an amino acid sequence, encoding an oxidoreductase, according to claim 1, that is ≥65%, identical to the amino acid sequence of SEQ ID NO:1 (Geobacillus sp. PA9), SEQ ID NO:3 (Thermus thermophilus), SEQ ID NO:4 (Streptomyces globisporus), SEQ ID NO:5 (Clostridium aminobutyricum), SEQ ID:6 (Burkholderai cepacia), SEQ ID NO:7 (Cupriavidus necator), SEQ ID NO:8 (Oscillatoria sp. PCC 6506), or SEQ ID NO:9 (Paraburkholderia phymatum),
wherein the polynucleotide has an amino acid exchange in one or more of positions 202, 203, 204, 205, 206, 207, 208, 209, 210, 211, 212, 213, 214 of SEQ ID NO:1, or at a corresponding position of the amino acid sequence of SEQ ID NO:3, SEQ ID NO:4, SEQ ID:5, SEQ ID NO:6, SEQ ID NO:7, SEQ ID NO:8, or SEQ ID NO:9.

3. The polynucleotide, encoding an amino acid sequence, encoding an oxidoreductase, according to claim 1, that is ≥90%, identical to the amino acid sequence of SEQ ID NO:1 (Geobacillus sp. PA9), SEQ ID NO:3 (Thermus thermophilus), SEQ ID NO:4 (Streptomyces globisporus), SEQ ID NO:5 (Clostridium aminobutyricum), SEQ ID:6 (Burkholderai cepacia), SEQ ID NO:7 (Cupriavidus necator), SEQ ID NO:8 (Oscillatoria sp. PCC 6506), SEQ ID NO:9 (Paraburkholderia phymatum), or SEQ ID NO:10 (Ralstonia pickettii),
wherein the polynucleotide has an amino acid exchange in one or more of positions 202, 203, 204, 205, 206, 207, 208, 209, 210, 211, 212, 213, 214 of SEQ ID NO:1, or at a corresponding position of the amino acid sequence of SEQ ID NO:3, SEQ ID NO:4, SEQ ID:5, SEQ ID NO:6, SEQ ID NO:7, SEQ ID NO:8, SEQ ID NO:9, or SEQ ID NO:10.

4. The polynucleotide encoding an amino acid sequence, encoding an oxidoreductase, according to claim 1, selected from
SEQ ID NO:1 (Geobacillus sp. PA9) with an amino acid exchange at one or more of the positions 202, 203, 204, 205, 206, 207, 208, 209, 210, 211, 212, 213, 214;
SEQ ID NO:3 (Thermus thermophilus) with an amino acid exchange at one or more of the positions 194, 195, 196, 197, 198, 199, 200, 201, 202, 203, 204, 205; or SEQ ID NO:21;
SEQ ID NO:4 (Streptomyces globisporus) with an amino acid exchange at one or more of the positions 212, 213, 214, 215, 216, 217, 218, 219, 220, 221, 222; or SEQ ID NO:22;
SEQ ID NO:5 (Clostridium aminobutyricum) with an amino acid exchange at one or more of the positions 202, 203, 204, 205, 206, 207, 208, 209, 210; or SEQ ID NO:23;
SEQ ID:6 (Burkholderai cepacia) with an amino acid exchange at one or more of the positions 202, 203, 204, 205, 206, 207, 208, 209, 210, 211, 212; or SEQ ID NO:24;
SEQ ID NO:7 (Cupriavidus necator) with an amino acid exchange at one or more of the positions 203, 204, 205, 206, 207, 208, 209, 210, 211, 212, 213; or SEQ ID NO:25;
SEQ ID NO:8 (Oscillatoria sp. PCC 6506) with an amino acid exchange at one or more of the positions 209, 210, 211, 212, 213, 214, 215, 216, 217; or SEQ ID NO:26;
SEQ ID NO:9 (Paraburkholderia phymatum) with an amino acid exchange at one or more of the positions 192, 193, 194, 195, 196, 197, 198, 199, 200, 201; or SEQ ID NO:27; or
SEQ ID NO:10 (Ralstonia pickettii) with an amino acid exchange at one or more of the positions 203, 204, 205, 206, 207, 208, 209, 210, 211, 212, 213, 214, 215, 216, 217 or SEQ ID NO:28.

5. The polynucleotide encoding an amino acid sequence, encoding an oxidoreductase, according to claim 1, that is ≥92% identical to the amino acid sequence of SEQ ID NO:11, SEQ ID NO:12, SEQ ID NO:13 or SEQ ID NO:14 wherein SEQ ID NO:11, SEQ ID NO:12, SEQ ID NO:13 and SEQ ID NO:14, at position 207, or at a corresponding position of the amino acid sequence, has a proteinogenic amino acid other than L-valine.

6. The polynucleotide encoding an amino acid sequence, encoding an oxidoreductase, according to claim 1, wherein the polynucleotide is a replicable nucleotide sequence encoding an enzyme 4-hydroxyphenylacetate 3-monooxygenase from microorganisms of a genus Geobacillus, wherein protein sequences encoded thereby contain a proteinogenic amino acid other than L-valine at a position corresponding to position 207 of SEQ ID NO:1.

7. The polynucleotide encoding an amino acid sequence, encoding an oxidoreductase, according to claim 1, wherein the amino acid sequence encoded thereby has, at the position 207 or a corresponding position, an amino acid which is selected from the group consisting of threonine, leucine, glutamine and glycine.

8. The polynucleotide encoding an amino acid sequence, encoding an oxidoreductase, according to claim 1, wherein the amino acid sequence encoded thereby
contain a proteinogenic amino acid other than L-threonine at position 206, or at a corresponding position of the amino acid sequence; or
contain a proteinogenic amino acid other than L-lysine at position 208, or at a corresponding position of the amino acid sequence.

9. The polynucleotide encoding an amino acid sequence, encoding an oxidoreductase, according to claim 1, that is ≥90%, identical to the amino acid sequence of SEQ ID NO:15, SEQ ID NO:16, SEQ ID NO:17, SEQ ID NO:18, SEQ ID NO:19, SEQ ID NO:20, SEQ ID NO:29, SEQ ID NO:30, SEQ ID NO:31, SEQ ID NO:32.

10. A vector comprising the polynucleotide according to claim 1.

11. The vector according to claim 10, which is suitable for replication in microorganisms of a genera Escherichia, Pseudomonas or Corynebacterium.

12. A polypeptide comprising an amino acid sequence encoded by the polynucleotide according to claim 1.

13. A microorganism of a genera Escherichia, Pseudomonas or Corynebacterium comprising the polynucleotide according to claim 1.

14. The microorganism according to claim 13, in which the polynucleotide is present in an overexpressed form.

15. The microorganism according to claim 13 having a capability of producing a fine chemical.

16. A fermentative process for producing a fine chemical comprising:
a) fermenting a microorganism comprising a polynucleotide encoding an amino acid sequence, encoding an oxidoreductase, that is ≥90%, identical to the amino acid sequence of SEQ ID NO:1 or SEQ ID NO:3 to SEQ ID NO:32 in a medium, and
b) accumulating the fine chemical in the medium, wherein a fermentation broth is obtained.

17. The fermentative process according to claim 16, wherein the fine chemical or a liquid or solid fine chemical-containing product is obtained from the fermentation broth and the fine chemical is L-DOPA.

18. A method of making L-DOPA comprising fermenting the microorganism according to claim 13.
